(19) United States
(12) Patent Application Publication
(10) Pub. No.: US 2005/0266093 A1
(43) Pub. Date: Dec. 1, 2005

(54) Nanogene therapy for cell proliferation disorders

(76) Inventor: Shyam S. Mohapatra, Tampa, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/117,169

(22) Filed: Apr. 27, 2005

Related U.S. Application Data
(60) Provisional application No. 60/565,756, filed on Apr. 27, 2004.

(51) Int. Cl. A61K 48/00; A61K 9/16; A61K 9/50

(52) U.S. Cl. 424/492; 514/44

(57) ABSTRACT

The present invention concerns particles comprising a chitin component, such as chitosan or a derivative thereof, associated with a polynucleotide encoding an interferon (IFN) molecule, 2-5′ oligoadenylate synthetase (2-5 AS), or a combination thereof. Preferably, the chitin component comprises chitosan or a derivative thereof. The particles of the invention are useful for delivery and expression of the interferon-encoding and/or 2-5 AS-encoding polynucleotide within a host in vitro or in vivo. The invention further concerns pharmaceutical compositions comprising particles of the invention and a pharmaceutically acceptable carrier, and a method for producing particles of the present invention. The present invention further pertains to a method of inducing apoptosis in a cancer cell, such as a lung cancer cell, by contacting a target cancer cell in vitro or in vivo with an effective amount of particles of the invention. In one embodiment, a therapeutically effective amount of particles are administered to target cancer cells within a patient in vivo, for treatment of cancer, such as lung cancer. The particles and therapeutic methods of the invention provide anti-metastatic and anti-cancer therapeutics for cancer patients, particularly lung cancer patients.

### CROSS-REFERENCE TO RELATED APPLICATION

This application claims benefit of U.S. Provisional Application Ser. No. 60/565,756, filed Apr. 27, 2004, which is hereby incorporated by reference herein in its entirety, including any figures, tables, nucleic acid sequences, amino acid sequences, and drawings.

### FIELD OF THE INVENTION

This invention pertains to particles including a chitin component, a polynucleotide encoding an interferon molecule, such as IFN-gamma (IFN-γ), or an interferon-inducible molecule such as 2′-5′ oligoadenylate synthetase (2-5 AS) or interferon regulatory factor (IRF-1), or a combination of any of the foregoing, and the use of such particles for treatment of cell proliferation disorders, such as lung cancer.

### BACKGROUND OF THE INVENTION

Lung cancer is one of the leading causes of death worldwide. Despite progress made in our understanding of the multiple risk factors associated with the development of lung cancer, and progress in developing novel approaches, this disease remains difficult to treat effectively. Lung cancer patients often present with locally advanced or disseminated disease. Their long-term survival is poor and such aggressive cancers are difficult to treat because of drug-induced toxicity. Non-viral plasmid DNA (pDNA)-mediated gene therapy, one of several new therapeutic approaches for lung cancer, provides a better alternative that is both safe and effective. Unlike viral vectors, which can induce an immune response with associated immunogenicity and systemic toxicity, a pDNA strategy combined with a chitosan-based nanoparticle (CBN) carrier system provides a unique approach to delivering genes by the mucosal route with limited toxicity and increased transgene expression, especially in target organs such as the lung (disclosed in Mohapatra et al., international publication WO 03/028759 A1 and Mohapatra et al., U.S. patent publication 2003-0068333-A1, which are each incorporated herein by reference in their entirety).

Lung tumor development and metastasis are complex processes that include transformation, proliferation, resistance to apoptosis, neovascularization, and metastatic spread (Antoniou, K. M. et al. Chest, 2003, 123:209-216). A number of gene products have been identified that play critical roles in these processes. Inhibition of metastasis is one of the most important therapeutic strategies in the treatment of lung cancer, since approximately 70% of lung cancer patients die from the metastatic disease even after a complete resection of primary tumor. Metastasis involves the disruption of extracellular matrix (ECM) adhesion, ECM degradation, cell cycle disregulation, and escape from apoptosis. Thus, protection from metastasis would have to block one or more of these processes.

A complex array of endocrine activities controls cell proliferation and death in the respiratory, gastrointestinal and urinary mucosa, which are major sites of tumor development. Interferons (IFNs) have received wide attention for their anti-cancer effects and are currently used for many cancers. The major oncologic indications of IFNs include melanoma, renal cell carcinoma, AIDS-related carposi sarcoma, follicular lymphoma, hairy cell leukemia and chronic myelogenous leukemia (Antoniou, K. M. et al. Chest, 2003, 123:209-216). Exogenous recombinant IFNs have a shorter half-life in vivo, and systemic administration at moderate to high doses may cause substantial adverse effects (Gutterman, J. U. PNAS, 1994, 91:1198-1205; Antoniou, K. M. et al. Chest, 2003, 123:209-216).

To overcome the limitations inherent with therapy using cytokines per se (cytokine proteins or polypeptides), several investigators have used transient gene expression therapy involving these genes. Separately, IFN-γ and IL-12 have each proven effective both as prophylactics and adjuncts in therapy against diverse human diseases (Mohapatra, S. S. Science, 1995, 269(5230):1499; Murray, H. W. Intensive Care Med, 1996, 22(Suppl 4):S456-S461). Oromucosal IFN therapy was found to be effective for antiviral and antitumoral activity (Okubo, T. et al. J Immunol, 1999, 162:4013-4017). However, mucosal administration of IFN-γ pDNA has not been studied.

The last decade has seen tremendous progress in gene expression technology. Several investigators have utilized a replication-deficient episomal adenovirus as a vehicle for transient gene expression. Adenoviral vectors are very efficient at transducing target cells in vitro and in vivo and permit transgene expression in a dose-dependent manner (Behera, A. K. et al. Hum Gene Ther., 2002, 13:1697-1709), but they do produce acute inflammation and an immune response to viral vector encoded antigens, which remain the major stumbling blocks to the application of adenovirus-mediated IFN-γ gene transfer for treating human diseases. Previous studies have demonstrated that the mucosal administration of pIFN-γ significantly decreased airway inflammation and airway hyper-responsiveness in a mouse model of grass allergic asthma. Adenoviral-mediated IFN-γ gene transfer effectively reversed established asthma in a BALB/c mouse model (Behera, A. K. et al. Hum Gene Ther., 2002, 13:1697-1709).

It has recently been shown that intranasally delivered pDNA encoding interferon gamma (IFN-γ) can be used as an antiviral treatment against respiratory syncytial virus infection (Mohapatra et al., U.S. Pat. No. 6,489,306). Further, IFN-γ is known to induce interferon response factor (IRF-1) and 2′5′ oligoadenylate synthetase (2-5 OAS), which also have antiviral properties (Behera, A. K. et al., JBC, 2002, 277(28):25601-25608; Mohapatra et al., U.S. patent publication 2004-0009152-A1; Mohapatra et al., international publication WO 03/092618 A2; which are each incorporated herein by reference in their entirety). Also, an IFN-γ producing plasmid encapsulated in a chitin-based nanoparticle, which has been referred to as “CIN”, has been shown to possess anti-inflammatory and apoptosis-inducing properties and to attenuate lung inflammation and airway hypereactivity (Kumar et al. Genet Vacc Ther, 2003, 1(1):3; Mohapatra, international publication WO 2004/074314 A2; which are each incorporated herein by reference in their entirety).

The present inventors reasoned that intranasally administered nanoparticles capable of de novo production of the IFN-γ may provide a novel means of prophylaxis and/or treatment for cancer, such as metastatic lung cancer. Research in the laboratory has identified the pIFN-γ as a potential lung cancer treatment based on its ability to induce significant apoptosis in cultured lung cancer cell lines. Also, CBN complexed p-DNA encoding pIFN-γ was found to completely abrogate the development of lung tumors in a nude mouse model of metastatic lung cancer.

Non-viral mediated gene expression using plasmid DNAs (pDNAs) has a number of advantages, including ease of preparation and use, stability, and room temperature storage (Hellerman, G. R. and Mohapatra, S. S. Gen Vacc &Ther, 2003, 1:1). They do not replicate in mammalian cells and do not integrate into host genomes, yet they can persist in host cells and express the cloned gene for a period of weeks to months. One problem associated with the pDNA approach is inefficient gene transfer in vivo, especially in slow and non-dividing cells such as epithelial cells (Mohapatra, S. S. Pediatr. Infect. Dis. J., 2003, 22(2 Suppl):S100-S103). CBNs protect pDNA from nuclease degradation and facilitate its entry into target cells. CBNs are prepared from chitosan, a biocompatible cationic polysaccharide from chitin extracted from crustacean shells, and have shown excellent potential for gene (Mao, H-Q. et al. J. Controlled Release, 2001, 70(3):399-421, which is incorporated herein by reference in its entirety) and controlled drug delivery. Chitosan is non-toxic, resistant to biodegradation, non-hemolytic, stimulates the immune system, is an anticoagulant, and has wound-healing and antimicrobial properties. Chitosan also increases transcellular and paracellular transport across the mucosal epithelium, thereby facilitating mucosal gene delivery. Another advantage of the use of CBNs for gene transport is their ability to target specific cells. Reduction of nonspecific interactions by shielding of net positive surface charges also improves targeting of CBNs.

### BRIEF SUMMARY OF THE INVENTION

In one aspect, the present invention concerns particles comprising a chitin component, which is associated with a polynucleotide encoding an interferon (IFN) or an IFN-inducible protein, such as 2′-5′ oligoadenylate synthatase (2-5 AS) or interferon regulatory factor (IRF-1). Preferably, the chitin component comprises chitosan or a derivative thereof. Optionally, the particles of the invention further comprise a lipid component and are referred to herein interchangeably as “chliposomes”, “chlipids”, “chitosan-lipid nanoparticles” or “CLNs”. The particles of the invention are useful for delivery and expression of the interferon-encoding and/or IFN-inducible molecule-encoding polynucleotide within a host in vitro or in vivo. The invention further concerns a method for producing particles of the present invention.

In some embodiments, the particles of the invention comprise a polynucleotide encoding an interferon selected from the group consisting of alpha-interferon, beta-interferon, gamma-interferon, omega-interferon, and lambda-interferon. In some embodiments, the particles of the invention comprise a polynucleotide encoding 2-5 AS or at least one catalytically active fragment thereof selected from the group consisting of the p40, p69, and p100 subunit. Such 2-5 AS subunits may be one or more splice variants, such as the 42 kDa, 46 kDa, 69 kDa, and/or 71 kDa variant. In some embodiments, the particles of the invention comprise a polynucleotide encoding IRF-1, or a biologically active fragment or homolog thereof.

In another aspect, the present invention concerns a pharmaceutical composition comprising particles comprising a chitin component and a polynucleotide encoding an interferon (IFN) molecule, an IFN-inducible molecule, or a combination thereof; and a pharmaceutically acceptable carrier. Optionally, the particles of the invention further comprise a lipid component. In one embodiment, the pharmaceutical composition is formulated for delivery through a mucosal route, such as the lungs.

In another aspect, the present invention concerns a method of treating a cell proliferation disorder by administering a therapeutically effective amount of particles to a patient in need thereof. Accordingly, a method of reducing cellular growth by administering a therapeutically effective amount of particles of the invention is contemplated, in order to reduce (partially or completely inhibit, prevent, or slow) uncontrolled cell growth. In one embodiment, an effective amount of particles are administered to a patient for treatment of cancer, such as lung cancer.

In another aspect, the present invention concerns a method of inducing apoptosis in a cancer cell, such as a lung cancer cell, by contacting a target cancer cell in vitro or in vivo with an effective amount of particles of the invention. In one embodiment, a therapeutically effective amount of particles are administered to target cancer cells within a patient in vivo, for treatment of cancer, such as lung cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an analysis of apoptosis using fluorescence microscopy in cells transfected with pIFN-γ. The micrographs show that IFN-gamma treatment of HEp-2 cells induces apoptosis.

FIG. 2 shows an immunoblot demonstrating the detection of p27kip expression and PARP cleavage in IFN-gamma treated HEp-2 cells with or without RSV infection.

FIG. 3 shows immunocytochemistry following pIFN-gamma treatment. BALB/c nude mice were injected with A549 cells (5×10⁶ cells/mouse) intravenously (i.v.) and one group treated with pIFN-gamma and another group with pVAX as control.

FIGS. 4A-4D show derivation and characterization of NG-042 nanoparticles. FIG. 4A shows synthesis and characterization of nanochitosan particles produced by proprietary method. The products were separated by capillary gel electrophoresis. The plot shows the separation of 4 low molecular weight components. The nanogene particles were then subject to analysis of size and zeta potential using a NiComp381 Zetasizer. Results are shown in FIG. 4B. The intensity weight distribution of NG042 particles showing their size of 155 nm, zeta potential=20.42. Atomic Force Microscopic analysis of Nanogene-042 particles showing oligomeric structure complexed with DNA (red arrows; upper line) is shown in FIG. 4C. FIG. 4D shows that lyophilzed and resuspended NG042 particles retain functionality at ambient temperatures of 23° to 55° C. Nanogene complexes of pGL3 (firefly luciferase, Promega) was lyophilized, reconstituted with water and treated for 24 hours at RT (23° C.), 42° C., 55° C. and −20° C. A549 cells were plated and transfected with the above complexes. Uptake and expression of DNA was allowed to occur for 24 hours. Luciferase activity was determined by using Promega's Dual Assay kit. Readings were normalized to relative luminiscence units (RLU) per mg protein.

FIGS. 5A-5C show characterization of NG044 particles. FIG. 5A shows that expression of nanoparticle-encapsulated EGFP gene continues in vivo until day 10. NG044 particles were complexed with DNA (5:1) encoding green fluorescent protein and administered intranasally to groups of mice (n=3). Mice were sacrificed on the indicated days and broncho-alveolar lavage cells were examined by fluorescent microscopy. FIG. 5B demonstrations the thermogelling property of NG044. NG044 forms a gel upon reacting with 2-glycerol phosphate, while NG042, another depolymerized chitosan, does not. To test the controlled release of gene expression, NG044 hydrogel was prepared using pEGFP plasmid DNA and PVP/glutaraldehyde for gel formation. The hydrogel was freeze-dried and the powder was resuspended in water (NG044 hydrogel) and given intranasally to groups of mice (n=4). Another group received NG044 with pEGFP without gelling (Control). Gene expression in the mouse lung was measured by EGFP expression in BAL cells 10 and 20 days after administration. Results are shown in FIG. 5C. The results at day 10 were similar (not shown) for control and hydrogel, whereas after 20 days mice given hydrogel continued EGFP show expression and no expression was detected in control mice.

### BRIEF DESCRIPTION OF THE SEQUENCES

SEQ ID NO: 1 is a nucleotide coding sequence (CDS) for the human 40 kDa splice variant of the 40/46 kDa subunit (“p40 subunit”) of 2′-5′ oligoadenylate synthetase (National Center for Biotechnology Information (NCBI) Accession Number NM_{—}016816).

SEQ ID NO: 2 is an amino acid sequence of the human 40 kDa splice variant of the 40/46 kDa subunit (“p40 subunit”) of 2′-5′ oligoadenylate synthetase (NCBI Accession Number NM_{—}016816).

SEQ ID NO: 3 is a nucleotide coding sequence (CDS) for the human 46 kDA splice variant of the 40/46 kDa subunit (“p40 subunit”) of 2′-5′ oligoadenylate synthetase (National Center for Biotechnology Information (NCBI) Accession Number NM_{—}016816).

SEQ ID NO: 4 is an amino acid sequence of the human 46 kDA splice variant of the 40/46 kDa subunit (“p40 subunit”) of 2′-5′ oligoadenylate synthetase (NCBI Accession Number NM_{—}016816).

SEQ ID NO: 5 is a nucleotide coding sequence (CDS) for the human 69 kDA splice variant of the 69/71 kDa subunit (“p69 subunit”) of 2′-5′ oligoadenylate synthetase (NCBI Accession Number NM_{—}002535).

SEQ ID NO: 6 is an amino acid sequence of the human 69% kDa splice variant of the 69/71 kDa subunit (“p69 subunit”) of 2′-5′ oligoadenylate synthetase (NCBI Accession Number NM_{—}002535).

SEQ ID NO: 7 is a nucleotide coding sequence (CDS) for the human 71 kDA splice variant of the 69/71 kDa subunit (“p69 subunit”) of 2′-5′ oligoadenylate synthetase (NCBI Accession Number NM_{—}002535).

SEQ ID NO: 8 is an amino acid sequence of the human 71 kDa splice variant of the 69/71 kDa subunit (“p69 subunit”) of 2′-5′ oligoadenylate synthetase (NCBI Accession Number NM_{—}002535).

SEQ ID NO: 9 is a nucleotide coding sequence (CDS) for the human 100 kDa subunit (“p100 subunit”) of 2′-5′ oligoadenylate synthetase (NCBI Accession Number AF063613).

SEQ ID NO: 10 is an amino acid sequence of the human 100 kDa subunit (“p100 subunit”) of 2′-5′ oligoadenylate synthetase (NCBI Accession Number AF063613).

SEQ ID NO: 11 is a nucleotide coding sequence (CDS) for the mouse homolog of the 2′-5′ oligoadenylate synthetase 40 kDa splice variant (p40 subunit) (NCBI Accession Number M33863).

SEQ ID NO: 12 is the amino acid sequence for the mouse homolog of the 2′-5′ oligoadenylate synthetase 40 kDa splice variant (p40 subunit) (NCBI Accession Number M33863).

SEQ ID NO: 13 is the human 2′-5′ oligoadenylate synthetase 40/46 kDa (p40 subunit) gene (NCBI Accession Number NM_{—}016816).

SEQ ID NO: 14 is the human 2′-5′ oligoadenylate synthetase 69/71 kDa (p69 subunit) gene (NCBI Accession Number NM_{—}002535).

SEQ ID NO: 15 is the human 2′-5′ oligoadenylate synthetase 100 kDa (p100 subunit) gene (NCBI Accession Number AF063613).

SEQ ID NO: 16 is the mouse homolog of the 2′-5′ oligoadenylate synthetase 40 kDa (p40 subunit) gene (NCBI Accession Number M33863).

SEQ ID NO: 17 is the nucleotide coding sequence (CDS) for human IFN-γ (NCBI Accession No: NM_{—}000639.

SEQ ID NO: 18 is the amino acid sequence for human IFN-γ (NCBI Accession No: NM_{—}000639.

SEQ ID NO:19 is the nucleotide coding sequence (CDS) for human interferon-beta (NCBI Accession No.: M25460).

SEQ ID NO:20 is the nucleotide coding sequence (CDS) for human interferon-beta-1 (NCBI Accession No.: M28622).

SEQ ID NO:21 is the nucleotide coding sequence (CDS) for a human interferon (NCBI Accession No.: L25664).

SEQ ID NO:22 is the nucleotide coding sequence (CDS) for human interferon-alpha (NCBI Accession No.: M54886 and M38682).

SEQ ID NO:23 is the nucleotide coding sequence (CDS) for human interferon-alpha-J1 (NCBI Accession No.: M34913).

SEQ ID NO:24 is the nucleotide coding sequence (CDS) for human interferon-omega-1 (NCBI Accession No.: X58822).

SEQ ID NO:25 is the nucleotide coding sequence (CDS) for human interleukin 28A (interferon, lambda 2; IL-28A) (NCBI Accession No.: NM_{—}172138).

SEQ ID NO:26 is the nucleotide coding sequence (CDS) for human interleukin 28B (interferon lambda 3; IL-28B) (NCBI Accession No.: AY336714).

SEQ ID NO:27 is the nucleotide coding sequence (CDS) for human interleukin 28C (interferon lambda 4; IL-28C) (NCBI Accession No.: AY336717).

SEQ ID NO:28 is the nucleotide coding sequence (CDS) for human interleukin 29 (interferon lambda 1; IL-29) (NCBI Accession No.: NM_{—}172140).

SEQ ID NO:29 is the nucleotide coding sequence (CDS) for a human interferon-like peptide (NCBI Accession No.: EE00870).

SEQ ID NO:30 is the nucleotide coding sequence (CDS) for a human interferon-like peptide (NCBI Accession No.: EE00871).

SEQ ID NO:31 is the nucleotide coding sequence (CDS) for a human interferon-regulatory factor 1 (IRF-1) (NCBI Accession No.: 002198).

### DETAILED DESCRIPTION OF THE INVENTION

The present invention concerns particles comprising a chitin component, such as chitosan or a derivative thereof, associated with a polynucleotide encoding an interferon (IFN) molecule or an IFN-inducible molecule, or a combination thereof. Preferably, the particles further comprise a control sequence operably-linked to the polynucleotide, which is capable of causing expression of the polynucleotide within a host in vitro or in vivo.

In certain embodiments, the interferon molecule encoded by the polynucleotide is Type I or Type II interferon, including those commonly designated as alpha-interferon, beta-interferon, gamma-interferon, and omega-interferon (also designated α-interferon, β-interferon, γ-interferon, and ω-interferon), and combinations thereof, including the consensus sequence for alpha-interferon. In some embodiments, the alpha-interferon is alpha₁ or alpha₂-interferon. In some embodiments, the interferon is interferon α-2b. Other interferons include interferon α-2β, a fusion interferon α-/2α-1, interferon α-2e, human α1 or α2 interferon.

In some embodiments, the interferon is a hybrid interferon. The construction of hybrid polynucleotides encoding combinations of different interferon subtypes (such as α and ε; α and β, and α and F) is disclosed in U.S. Pat. Nos. 4,414,150; 4,456,748; and 4,678,751, each of which are incorporated herein by reference in their entirety. U.S. Pat. Nos. 4,695,623; 4,897,471; and 5,831,062, which are incorporated herein by reference in their entirety, disclose novel human leukocyte interferon polypeptides having amino acid sequences that include common or predominant amino acids found at each position among naturally-occurring alpha interferon subtype polypeptides and are referred to as consensus human leukocyte interferon. In one embodiment of the invention, the hybrid interferon is interferon α2α1.

In one embodiment, the interferon is an interferon-α. Recombinant interferon alphas, for instance, have been cloned and expressed in E. coli by several groups (e.g., Weissmann et al., Science, 1980, 209:1343-1349; Sreuli et al., Science, 1980, 209:1343-1347; Goeddel et al., Nature, 1981, 290:20-26; Henco et al., J. Mol. Biol., 1985, 185:227-260, each of which are incorporated herein by reference in their entirety). In some embodiments, the interferon is a human interferon alpha. In some embodiments, the interferon alpha is interferon alpha 2a or 2b.

The term “interferon” as used herein is intended to include all classes and subclasses of interferon, and deletion, insertion, or substitution variants, as well as “interferon-like” molecules such as interleukin 15 (IL-15), interleukin 28A (interferon lambda2; IL-28A), interleukin 28B (IL-28B), interleukin 28C (IL-28C), interleukin 29 (interferon lambda1; IL-29), and synthetic interferon-like peptides (e.g., NCBI accession nos. E00871 and E00870). In one embodiment, the interferon-encoding polynucleotide, or its polypeptide product, is the interferon-alpha-encoding polynucleotide or its polypeptide product. In some embodiments, the interferon-encoding polynucleotide of the particle, or its polypeptide, is the human nucleotide or amino acid sequence. The human interferon alphas, for example, are a family of proteins including at least 24 subspecies (Zoon, K. C., Interferon, 1987, 9:1, Gresser, I., ed., Academic Press, NY). The interferon alphas were originally described as agents capable of inducing an antiviral state in cells but are now known as pleiotropic lymphokines affecting many functions of the immune system (Openakker et al., Experimentia, 1989, 45:513). In some embodiments, the interferon alpha is interferon alpha 2a or 2b (see, for example, WO 91/18927, which is incorporated by reference herein in its entirety), although any interferon alpha may be used. Nucleotide sequences encoding the exemplified interferons interferon-gamma; interferon-beta; interferon-beta-1; interferon; interferon-alpha; interferon-alpha-J1; interferon omega-1; interleukin 28A; interleukin 28B; interleukin 28C, interleukin 29; and interferon-like peptides are listed as SEQ ID NOs: 17 and 19-30. Particles of the invention may contain one or more of these polynucleotides or degenerate sequences encoding the same polypeptides, for example

The interferon-encoding polynucleotide may encode gamma-interferon (IFN-γ), among others. IFN-γ is a 14-18 kDalton 143 amino acid glycosylated protein that is a potent multifunctional cytokine. As used herein, “interferon-gamma”, “IFN-gamma”, “interferon-γ”, and “IFN-γ refer to IFN-γ protein, biologically active fragments of IFN-γ, and biologically active homologs of “interferon-gamma” and “IFN-γ”, such as mammalian homologs. These terms include IFN-γ-like molecules. An “IFN-γ-like molecule” refers to polypeptides exhibiting IFN-γ-like activity when the polynucleotide encoding the polypeptide is expressed, as can be determined in vitro or in vivo. For purposes of the subject invention, IFN-γ-like activity refers to those polypeptides having one or more of the functions of the native IFN-γ cytokine disclosed herein (such as induction of apoptosis). Fragments and homologs of IFN-γ retaining one or more of the functions of the native IFN-γ cytokine, such as those disclosed herein, is included within the meaning of the term “IFN-γ”. In addition, the term includes a nucleotide sequence which through the degeneracy of the genetic code encodes a similar peptide gene product as IFN-γ and has the IFN-γ activity described herein. For example, a homolog of “interferon-gamma” and “IFN-γ” includes a nucleotide sequence which contains a “silent” codon substitution (e.g., substitution of one codon encoding an amino acid for another codon encoding the same amino acid) or an amino acid sequence which contains a “silent” amino acid substitution (e.g., substitution of one acidic amino acid for another acidic amino acid).

An exemplified nucleotide sequence encodes human IFN-γ (Accession No: NM_{—}000639, NCBI database, which is hereby incorporated by reference in its entirety):

| (SEQ ID NO: 17) | |
|---|---|
| 1 | tgaagatcag ctattagaag agaaagatca gttaagtcct ttggacctga tcagcttgat |
| 61 | acaagaacta ctgatttcaa cttctttggc ttaattctct cggaaacgat gaaatataca |
| 121 | agttatatct tggcttttca gctctgcatc gttttgggtt ctcttggctg ttactgccag |
| 181 | gacccatatg taaaagaagc agaaaacctt aagaaatatt ttaatgcagg tcattcagat |
| 241 | gtagcggata atggaactct tttcttaggc attttgaaga attggaaaga ggagagtgac |
| 301 | agaaaaataa tgcagagcca aattgtctcc ttttacttca aactttttaa aaactttaaa |
| 361 | gatgaccaga gcatccaaaa gagtgtggag accatcaagg aagacatgaa tgtcaagttt |
| 421 | ttcaatagca acaaaaagaa acgagatgac ttcgaaaagc tgactaatta ttcggtaact |
| 481 | gacttgaatg tccaacgcaa agcaatacat gaactcatcc aagtgatggc tgaactgtcg |
| 541 | ccagcagcta aaacagggaa gcgaaaaagg agtcagatgc tgtttcaagg tcgaagagca |
| 601 | tcccagtaat ggttgtcctg cctgcaatat ttgaatttta aatctaaatc tatttattaa |
| 661 | tatttaacat tatttatatg gggaatatat ttttagactc atcaatcaaa taagtattta |
| 721 | taatagcaac ttttgtgtaa tgaaaatgaa tatctattaa tatatgtatt atttataatt |
| 781 | cctatatcct gtgactgtct cacttaatcc tttgttttct gactaattag gcaaggctat |
| 841 | gtgattacaa ggctttatct caggggccaa ctaggcagcc aacctaagca agatcccatg |
| 901 | ggttgtgtgt ttatttcact tgatgataca atgaacactt ataagtgaag tgatactatc |
| 961 | cagttactgc cggtttgaaa atatgcctgc aatctgagcc agtgctttaa tggcatgtca |
| 1021 | gacagaactt gaatgtgtca ggtgaccctg atgaaaacat agcatctcag gagatttcat |
| 1081 | gcctggtgct tccaaatatt gttgacaact gtgactgtac ccaaatggaa agtaactcat |
| 1141 | ttgttaaaat tatcaatatc taatatatat gaataaagtg taagttcaca act |

(SEQ ID NO: 18)
MKYTSYILAFQLCIVLGSLGCYCQDPYVKEAENLKKYFNAGHSDVADNGTLFLGILKNWKEESDRKIMQ
SQIVSFYFKLFKNFKDDQSIQKSVETIKEDMNVKFFNSNKKKRDDFEKLTNYSVTDLNVQRKAIHELIQ
VMAELSPAAKTGKRKRSQMLFQ GRRASQ

U.S. Pat. Nos. 5,770,191 and 6,120,762, which are incorporated herein by reference in their entirety, describe several C-terminal fragments of IFN-gamma that may be encoded by the polynucleotide(s) carried by the particles of the invention. Other interferons that may be encoded by polynucleotides within the particles of the invention are described in U.S. patent publications 2005-0054052-A1; 2005-0054053-A1; 2005-0025742-A1; and 2005-0084478-A1; which are incorporated herein by reference in their entirety.

Interferon regulatory factor-1 (IRF-1) is an interferon-inducible molecule (e.g., an interferon-stimulated gene product) (Pizzoferrato, F. et al., Cancer Res., 2004, 64(22):8381-8388; Pack, S. Y. et al., Eur. J. Biochem., 2004, 271(21):4222-4228). The nucleotide sequence encoding human IRF-1 is provided herein as SEQ ID NO: 31. Particles of the invention may contain this polynucleotide, degenerate sequences encoding the same polypeptide, or biologically active fragments thereof, for example.

2′5′ oligoadenylate synthetase (2-5 AS) is an interferon-inducible molecule. In some embodiments, the particles of the invention comprise a polynucleotide encoding 2-5 AS or at least one catalytically active fragment thereof selected from the group consisting of the p40, p69, and p100 subunit. Such 2-5 AS subunits may be one or more splice variants, such as the 42 kDa, 46 kDa, 69 kDa, and/or 71 kDa variant. For example, the particles can comprise one or more nucleotide sequences encoding polypeptides comprising one more amino acid sequences set forth herein as SEQ ID NOs: 2, 4, 5, 6, 8, 10, 12, 13, 14, 15, or 16, or catalytically active fragments of these amino acids. In some embodiments, the particles comprise one or more nucleotide sequences selected from the group consisting of SEQ ID NOs: 1, 3, 5, 7, 9, 11, 13, 14, 15, 16, and 18, or a catalytically active fragment thereof.

As indicated above, the particle used in the compositions and methods of the invention comprise polynucleotides encoding an interferon, an interferon-inducible molecule, or both. For example, the particle can contain a polynucleotide encoding an interferon and 2-5 AS; encoding an interferon and IRF-1; encoding 2-5 AS and IRF-1; or encoding an interferon, 2-5 AS, and IRF-1. Combinations of an interferon and interferon-inducible molecules can be encoded by polynucleotides within a single particle or multiple different particles.

The nucleotide sequences encoding interferon and/or an interferon-inducible molecule used in the subject invention include “homologous” or “modified” nucleotide sequences. Modified nucleic acid sequences will be understood to mean any nucleotide sequence obtained by mutagenesis according to techniques well known to persons skilled in the art, and exhibiting modifications in relation to the normal sequences. For example, mutations in the regulatory and/or promoter sequences for the expression of a polypeptide that result in a modification of the level of expression of a polypeptide according to the invention provide for a “modified nucleotide sequence”. Likewise, substitutions, deletions, or additions of nucleic acids to the polynucleotides of the invention provide for “homologous” or “modified” nucleotide sequences. In various embodiments, “homologous” or “modified” nucleic acid sequences have substantially the same biological or serological activity as the native (naturally occurring) interferon and/or interferon-inducible polypeptide. A “homologous” or “modified” nucleotide sequence will also be understood to mean a splice variant of the polynucleotides of the instant invention or any nucleotide sequence encoding a “modified polypeptide” as defined below.

A homologous nucleotide sequence, for the purposes of the present invention, encompasses a nucleotide sequence having a percentage identity with the bases of the nucleotide sequences of between at least (or at least about) 20.00% to 99.99% (inclusive). The aforementioned range of percent identity is to be taken as including, and providing written description and support for, any fractional percentage, in intervals of 0.01%, between 20.00% and 99.99%. These percentages are purely statistical and differences between two nucleic acid sequences can be distributed randomly and over the entire sequence length.

In various embodiments, homologous sequences exhibiting a percentage identity with the bases of the nucleotide sequences of the present invention can have 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity with the polynucleotide sequences of the instant invention. Homologous nucleic acid sequences and amino acid sequences include mammalian homologs of the human interferon and/or interferon-inducible molecule nucleic acid sequences and amino acid sequences, including homologs of biologically active fragments, such as biologically active subunits.

Both protein and nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman Proc. Natl. Acad. Sci. USA, 1988, 85(8):2444-2448; Altschul et al. J. Mol. Biol., 1990, 215(3):403-410; Thompson et al. Nucleic Acids Res., 1994, 22(2):4673-4680; Higgins et al. Methods Enzymol., 1996, 266:383-402; Altschul et al. J. Mol. Biol., 1990, 215(3):403-410; Altschul et al. Nature Genetics, 1993, 3:266-272).

Nucleotide sequences encoding polypeptides with enhanced interferon activity or interferon-inducible molecule activity (such as 2-5 AS catalytic activity and/or IRF-1 activity) can be obtained by “gene shuffling” (also referred to as “directed evolution”, and “directed mutagenesis”), and used in the compositions and methods of the present invention. Gene shuffling is a process of randomly recombining different sequences of functional genes (recombining favorable mutations in a random fashion) (U.S. Pat. Nos. 5,605,793; 5,811,238; 5,830,721; and 5,837,458). Thus, protein engineering can be accomplished by gene shuffling, random complex permutation sampling, or by rational design based on three-dimensional structure and classical protein chemistry (Cramer et al., Nature, 391:288-291, 1998; and Wulff et al., The Plant Cell, 13:255-272, 2001) Identity and similarity of related nucleic acid molecules and polypeptides can be readily calculated by known methods. Such methods include, but are not limited to, those described in Computational Molecular Biology, Lesk, A. M., ed., Oxford University Press, New York, 1988; York (1988); Biocomputing: Informatics and Genome Projects, Smith, D. W., ed., Academic Press, New York, 1993;York (1993); Computer Analysis of Sequence Data, Part 1, Griffin, A. M., and Griffin, H. G., eds., Humana Press, New Jersey, 1994;Jersey (1994); Sequence Analysis in Molecular Biology, von Heinje, G., Academic Press, 1987; Press (1987); Sequence Analysis Primer, Gribskov, M. and Devereux, J., eds., M. Stockton Press, New York, 1991; York (1991); and Carillo et al., SIAM J. Applied Math., 1988, 48:1073.

The particles, methods, and compositions of the present invention can utilize biologically active fragments of nucleic acid sequences encoding interferon and/or interferon-inducible molecules. Representative fragments of the polynucleotide sequences according to the invention will be understood to mean any polynucleotide fragment having at least 8 or 9 consecutive nucleotides, preferably at least 12 consecutive nucleotides, and still more preferably at least 15 or at least 20 consecutive nucleotides of the sequence from which it is derived. The upper limit for such fragments is the total number of nucleotides found in the full-length sequence (or, in certain embodiments, of the full length open reading frame (ORF) identified herein).

In other embodiments, fragments can comprise consecutive nucleotides of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, and up to one nucleotide less than the full-length interferon and/or 2-5 AS coding sequences (or, in some embodiments, up to the full length of nucleotides in the open reading frame (ORF)).

In some embodiments, fragments comprise biologically active subunits (such as the p40 subunit of 2-5 AS (e.g., 40 kDa, 42 kDa, 46 kDa, or other splice variant), p69 subunit of 2-5 AS (e.g., 69 kDa, 71 kDa, or other splice variant), p100 subunit of 2-5 AS, or combinations thereof).

It is also well known in the art that restriction enzymes can be used to obtain biologically active fragments of nucleic acid sequences, such as those encoding interferon and/or interferon-inducible molecules. For example, Bal31 exonuclease can be conveniently used for time-controlled limited digestion of DNA (commonly referred to as “erase-a-base” procedures). See, for example, Maniatis et al. (1982) Molecular Cloning: A Laboratory Manual, Cold Spring Harbor Laboratory, New York; Wei et al. [1983] J. Biol. Chem. 258: 13006-13512.

Optionally, each particle of the invention further comprises a lipid component that is complexed with the chitin and polynucleotide components of the particle. Since efficient gene expression in vivo requires both complex formation for cell uptake and prevention of nucleotide degradation and complex dissociation for transcription by RNA polymerase, a combination of a chitin component and lipid component (such as chitosan and liposomes, respectively) may lead to increased gene delivery and expression in vivo. Therefore, this embodiment of the particle combines these two different carrier systems (also referred to herein interchangeably as “chliposomes”, “chlipids”, “chitosan-lipid nanoparticles” or “CLNs”) to significantly increase polynucleotide transfection and expression. Preferably, the components of the chlipid are oriented such that the polynucleotide is surrounded by a lipid monolayer, with polynucleotide-lipid inverted cylindrical micelles arranged in a hexagonal lattice. Methods for producing CLNs containing polynucleotides encoding interferon-gamma are described in Mohapatra et al. international publication WO 2004/074314 A2, which is hereby incorporated herein in its entirety.

The present invention further includes a method for producing the particles of the invention by mixing (e.g., complexing) a polynucleotide and a chitin component, such as chitosan or a chitosan derivative, to form a particle comprising a binary complex of the polynucleotide and the chitin component. Optionally, the method further comprises mixing (complexing) a lipid with the polynucleotide and chitin component to form a particle (CLN) comprising a multiplex of the polynucleotide, the chitin component, and the lipid. Typically, the particles of the present invention range in size from the nanometer range (e.g., less than one micrometer; nanoparticles) to the micrometer size range (e.g., about one micrometer or larger). Methods for producing chitosan-based DNA particles are described in Mohapatra, S. S. Pediatr. Infect. Dis. J., 2003, 22(2 suppl.):S100-S103: Kumar, M. et al., Hum. Gene Ther., 2002, 13(12):1415-1425; Kumar et al., Genetic Vaccines and Therapy, 2003, 1:3; and Mohapatra et al., international publication no. WO 2004/074314 A2; each of which are incorporated herein by reference in their entirety.

The type of reaction vessel or substrate utilized for producing the particles of the present invention, or the size of the vessel or substrate, is not critical. Any vessel or substrate capable of holding or supporting the reactants so as to allow the reaction to take place can be used. It should be understood that, unless expressly indicated to the contrary, the terms “adding”, “contacting”, “mixing”, “reacting”, “combining” and grammatical variations thereof, are used interchangeably to refer to the mixture of reactants of the method of the present invention (such as plasmid DNA or a non-polynucleotide agent such as chitosan or a chitosan derivative, lipid, and so forth), and the reciprocal mixture of those reactants, one with the other (i.e., vice-versa), in any order.

It will be readily apparent to those of ordinary skill in the art that a number of general parameters can influence the efficiency of transfection or polynucleotide delivery. These include, for example, the concentration of polynucleotide to be delivered, the concentration of the chitin component (such as chitosan or a chitosan derivative), and the concentration of lipid (for chlipids of the present invention). For in vitro delivery, the number of cells transfected, the medium employed for delivery, the length of time the cells are incubated with the particles of the invention, and the relative amount of particles can influence delivery efficiency. For example, a 1:5 ratio of polynucleotide to lipid, 1:5 ratio of polynucleotide to chitosan, and 20% serum is suitable. These parameters can be optimized for particular cell types and conditions. Such optimization can be routinely conducted by one of ordinary skill in the art employing the guidance provided herein and knowledge generally available to those skilled in the art. It will also be apparent to those of ordinary skill in the art that alternative methods, reagents, procedures and techniques other than those specifically detailed herein can be employed or readily adapted to produce the particles and compositions of the invention. Such alternative methods, reagents, procedures and techniques are within the spirit and scope of this invention.

In accordance with the present invention, the polynucleotides carried by the particles are conjugated with a chitin component, such as chitosan or chitosan derivatives. For example, DNA chitosan nanospheres can be generated, as described by Roy, K. et al. (Nat Med, 1999, 5:387). Chitosan allows increased bioavailability of the nucleic acid sequences because of protection from degradation by serum nucleases in the matrix and thus has great potential as a mucosal gene delivery system. Chitosan also has many beneficial effects, including anticoagulant activity, wound-healing properties, and immunostimulatory activity, and is capable of modulating immunity of the mucosa and bronchus-associated lymphoid tissue.

The term “chitosan”, as used herein, will be understood by those skilled in the art to include all derivatives of chitin, or poly-N-aceryl-D-glucosamine (including all polyglucosamine and oligomers of glucosamine materials of different molecular weights), in which the greater proportion of the N-acetyl groups have been removed through hydrolysis. Generally, chitosans are a family of cationic, binary hetero-polysaccharides composed of (1→4)-linked 2-acetamido-2-deoxy-β-D-glucose (GlcNAc, A-unit) and 2-amino-2-deoxy-β-D-glucose, (GlcN; D-unit) (Varum K. M. et al., Carbohydr. Res., 1991, 217:19-27; Sannan T. et al., Macromol. Chem., 1776, 177:3589-3600). Preferably, the chitosan has a positive charge. Chitosan, chitosan derivatives or salts (e.g., nitrate, phosphate, sulphate, hydrochloride, glutamate, lactate or acetate salts) of chitosan may be used and are included within the meaning of the term “chitosan”. As used herein, the term “chitosan derivatives” are intended to include ester, ether or other derivatives formed by bonding of acyl and/or alkyl groups with OH groups, but not the NH₂ groups, of chitosan. Examples are O-alkyl ethers of chitosan and O-acyl esters of chitosan. Modified chitosans, particularly those conjugated to polyethylene glycol, are included in this definition. Low and medium viscosity chitosans (for example CL113, G210 and CL110) may be obtained from various sources, including PRONOVA Biopolymer, Ltd. (UK); SEIGAGAKU America Inc. (Maryland, USA); MERON (India) Pvt, Ltd. (India); VANSON Ltd. (Virginia, USA); and AMS Biotechnology Ltd. (UK). Suitable derivatives include those which are disclosed in Roberts, Chitin Chemistry, MacMillan Press Ltd., London (1992). Optimization of structural variables such as the charge density and molecular weight of the chitosan for efficiency of polynucleotide delivery and expression is contemplated and encompassed by the present invention.

The chitosan (or chitosan derivative or salt) used preferably has a molecular weight of 4,000 Dalton or more, preferably in the range 25,000 to 2,000,000 Dalton, and most preferably about 50,000 to 300,000 Dalton. Chitosans of different low molecular weights can be prepared by enzymatic degradation of chitosan using chitosanase or by the addition of nitrous acid. Both procedures are well known to those skilled in the art and are described in various publications (Li et al., Plant Physiol. Biochem., 1995, 33: 599-603; Allan and Peyron, Carbohydrate Research, 1995, 277:257-272; Damard and Cartier, Int. J. Biol. Macromol., 1989, 11: 297-302). Preferably, the chitosan is water-soluble and may be produced from chitin by deacetylation to a degree of greater than 40%, preferably between 50% and 98%, and more preferably between 70% and 90%.

The lipid component utilized for the particles, compositions, and methods of the present invention is preferably a phospholipid or cationic lipid. Cationic lipids are amphipathic molecules, containing hydrophobic moieties such as cholesterol or alkyl side chains and a cationic group, such as an amine. Phospholipids are amphipathic molecules containing a phosphate group and fatty acid side chains. Phospholipids can have an overall negative charge, positive charge, or neutral charge, depending on various substituents present on the side chains. Typical phospholipid hydrophilic groups include phosphatidyl choline, phosphatidylglycerol, and phosphatidyl ethanolamine moieties. Typical hydrophobic groups include a variety of saturated and unsaturated fatty acid moieties. The lipids used in the present invention include cationic lipids that form a complex with the genetic material (e.g., polynucleotide), which is generally polyanionic, and the chitosan or chitosan derivative. The lipid may also bind to polyanionic proteoglycans present on the surface of cells. The cationic lipids can be phospholipids or lipids without phosphate groups.

A variety of suitable cationic lipids are known in the art, such as those disclosed in International Publication No. WO 95/02698, the disclosure of which is herein incorporated by reference in its entirety. Exemplified structures of cationic lipids useful in the particles of the present invention are provided in Table 1 of International Publication No. WO 95/02698. Generally, any cationic lipid, either monovalent or polyvalent, can be used in the particles, compositions and methods of the present invention. Polyvalent cationic lipids are generally preferred. Cationic lipids include saturated and unsaturated allyl and alicyclic ethers and esters of amines, amides or derivatives thereof. Straight-chain and branched alkyl and alkene groups of cationic lipids can contain from 1 to about 25 carbon atoms. Preferred straight-chain or branched alkyl or alkene groups have six or more carbon atoms. Alicyclic groups can contain from about 6 to 30 carbon atoms. Preferred alicyclic groups include cholesterol and other steroid groups. Cationic lipids can be prepared with a variety of counterions (anions) including among others: chloride, bromide, iodide, fluoride, acetate, trifluoroacetate, sulfate, nitrite, and nitrate.

Transfection efficiency can be increased by using a lysophosphatide in particle formation. Preferred lysophosphatides include lysophosphatidylcholines such as I-oleoyllysophosphatidylcholine and lysophosphatidylethanolamines. Well known lysophosphatides which may be used include DOTMA (dioleyloxypropyl trimethylammonium chloride/DOPE (i.e., LIPOFECTIN, GIBCO/BRL, Gaithersburg, Md.), DOSPA, (dioleyloxy sperminecarboxamidoethyl dimethylpropanaminium trifuoroacetate)/DOPE (i.e., LIPOFECTAMINE), LIPOFECTAMINE 2000, and DOGS (dioctadecylamidospermine) (i.e., TRANSFECTAM), and are all commercially available. Additional suitable cationic lipids structurally related to DOTMA are described in U.S. Pat. No. 4,897,355, which is herein incorporated by reference in its entirety.

TRANSFECTAM belongs to a group of cationic lipids called lipopolamines (also referred to as second-generation cationic lipids) that differ from the other lipids used in gene transfer mostly by their spermine head group. The polycationic spermine head group promotes the formation of lipoplexes with better-defined structures (e.g., 50 to 100 nm) (Remy J. S. et al., “Gene Transfer with Lipospermines and Polyethylenimines”, Adv. Drug Deliv. Rev., 1998, 30:85-95).

Another useful group of cationic lipids related to DOTMA and DOTAP that may be utilized are commonly called DORI-ethers or DORI-esters, such as (DL-1-O-oleyl-2-oleyl-3-dimethylaminopropyl-β-hydroxyethylammonium or DL-1-oleyl-2-O oleyl-3-dimethylaminopropyl-β-hydroxyethylammonium). DORI lipids differ from DOTMA and DOTAP in that one of the methyl groups of the trimethylammonium group is replaced with a hydroxyethyl group. The oleoyl groups of DORI lipids can be replaced with other alkyl or alkene groups, such as palmitoyl or stearoyl groups. The hydroxyl group of the DORI-type lipids can be used as a site for further functionalization, for example for esterification to amines, like carboxyspermine. Additional cationic lipids which can be employed in the particles, compositions, and methods of the present invention include those described in International Publication No. WO 91/15501, which is herein incorporated by reference in its entirety. Cationic sterol derivatives, like 3 β [N—(N′,N′-dimethylaminoethane)carbamoyl] cholesterol (DC-Chol) in which cholesterol is linked to a trialkyammonium group, can also be employed in the present invention. DC-Chol is reported to provide more efficient transfection and lower toxicity than DOTMA-containing liposomes for some cell lines. DC-Chol polyamine variants such as those described in International Publication No. WO 97/45442 may also be used. Polycationic lipids containing carboxyspermine are also useful in the delivery vectors or complexes of this invention. EP-A-304111 describes carboxyspermine containing cationic lipids including 5-carboxyspermylglycine dioctadecyl-amide (DOGS), as referenced above, and dipalmitoylphosphatidylethanolamine 5-carboxyspermylamide (DPPES). Additional cationic lipids can be obtained by replacing the octadecyl and palmitoyl groups of DOGS and DPPES, respectively, with other alkyl or alkene groups. Cationic lipids can optionally be combined with non-cationic co-lipids, preferably neutral lipids, to form the chlipids of the invention. One or more amphiphilic compounds can optionally be incorporated in order to modify the particle's surface property.

Suitable cationic lipids include esters of the Rosenthal Inhibitor (RI) (DL-2,3-distearoyloxypropyl(dimethyl)-β-hydroxyethylammoniumbromide), as described in U.S. Pat. No. 5,264,618, the contents of which is hereby incorporated by reference in its entirety. These derivatives can be prepared, for example, by acyl and alkyl substitution of 3-dimethylaminopropane diol, followed by quaternization of the amino group. Analogous phospholipids can be similarly prepared.

The polynucleotides (and particles containing them) are administered and dosed in accordance with good medical practice, taking into account the clinical condition of the individual patient, the site and method of administration, scheduling of administration, patient age, sex, body weight, and other factors known to medical practitioners. The therapeutically or pharmaceutically “effective amount” for purposes herein is thus determined by such considerations as are known in the art. A therapeutically or pharmaceutically effective amount of polynucleotide (such as an IFN-encoding and/or IFN-inducible molecule-encoding polynucleotide) is that amount necessary to provide an effective amount of the polynucleotide, or the corresponding polypeptide(s) when expressed in vivo. An effective amount of an agent, such as a polynucleotide or non-polynucleotide agent, or particles comprising such polynucleotide or non-polynucleotide agents, can be an amount sufficient to prevent, treat, reduce and/or ameliorate the symptoms and/or underlying causes of any cell proliferation disorder, such as lung cancer. In some instances, an “effective amount” is sufficient to eliminate the symptoms of the pathologic condition and, perhaps, overcome the condition itself. In the context of the present invention, the terms “treat” and “therapy” and the like refer to alleviate, slow the progression, prophylaxis, attenuation, or cure of an existing condition. The term “prevent”, as used herein, refers to putting off, delaying, slowing, inhibiting, or otherwise stopping, reducing, or ameliorating the onset of such conditions. The therapeutic methods of the invention include prevention and/or treatment of a cell proliferation disorder.

In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

In one embodiment, the cells or subject to which the particles of the invention are administered is not suffering from an RNA virus infection, such as those disclosed in Mohapatra et al., international publication WO 03/092618 A2 and U.S. patent publication 2004-0009152-A1, which are incorporated herein by reference in their entirety. In another embodiment, the cells or subject to which the particles of the invention are administered is not suffering from a respiratory RNA virus infection. In another embodiment, the cells or subject to which the particles of the invention are administered is not suffering from a respiratory syncytial virus (RSV) infection.

Following administration of particles to a subject, the subject's physiological condition can be monitored in various ways well known to the skilled practitioner familiar with the hallmarks of cancer progression, or alternatively by monitoring the effects of administration of the particles on the amount and/or biological activity of the interferon and/or interferon-inducible molecule in vivo. Optionally, the therapeutic methods of the invention include identifying a subject suffering from a cell proliferation disorder, such as lung cancer or other cancer. Identification of the subject may include medical diagnosis of the disorder by a licensed clinician.

Mammalian species which benefit from the disclosed particles, compositions, and methods include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales.

As used herein, the term “patient”, “subject”, and “host” are used herein interchangeably and intended to include such human and non-human mammalian species and cells of those species. For example, the term “host” includes one or more host cells, which may be prokaryotic (such as bacterial cells) or eukaryotic cells (such as human or non-human mammalian cells), and may be in an in vivo or in vitro state. After particles of the invention are administered to cells in vitro, the cells may be administered to a subject. For example, the particles of the invention can be administered to a subject's cells ex vivo, followed by administration of the cells to the subject. In those cases wherein the polynucleotide utilized is a naturally occurring nucleic acid sequence, the polynucleotide encoding the polypeptide product can be administered to subjects of the same species or different species from which the nucleic acid sequence naturally exists, for example. When the subject is a human or the target cells are human, it is preferred that polynucleotides encoding human interferons and/or interferon-inducible molecules are utilized. However, mammalian homologs may also be used, for example.

The particles of the present invention (and compositions containing them) can be administered to a subject by any route that results in delivery and/or expression of the polynucleotide (such as plasmid DNA) or delivery of other non-polynucleotide agents carried by the particles at the desired site or sites. For example, the particles can be administered intravenously (I.V.), intramuscularly (I.M.), subcutaneously (S.C.), intradermally (I.D.), orally, intranasally, etc.

Examples of intranasal administration can be by means of a spray, drops, powder or gel and also described in U.S. Pat. No. 6,489,306, which is incorporated herein by reference in its entirety. One embodiment of the present invention is the administration of the invention as a nasal spray. Alternate embodiments include administration through any oral or mucosal routes such as oral, sublingual, intravaginal or intraanal administration, and even eye drops. However, other means of drug administrations such as subcutaneous, intravenous, and transdermal are well within the scope of the present invention.

In various embodiments, the cell proliferation disorder may be cancer of a mucous membrane, such as adenocarcinoma or other cancer of the lung, respiratory tract, stomach, epithelium, etc. As used herein, a “lung cancer” includes either a primary lung tumor (for example, bronchogenic carcinoma or bronchial carcinoid) or a metastasis from a primary tumor of another organ or tissue (for example, breast, colon, ovary, prostate, kidney, thyroid, stomach, peritoneum, cervix, rectum, testis, bone, or melanoma).

In preferred embodiments, for cell proliferation disorders of the respiratory tract such as the lung, the particles of the invention are administered through inhalation in a form such as an aerosol, a nebula, a mist, an atomized sample, liquid drops, etc. The particles are preferably delivered to the target respiratory tract tissue with a pharmacokinetic profile that results in the delivery of an effective dose of the polynucleotide carried by the particles. In preferred embodiments, at least 1%, more preferably at least 5%, even more preferably at least 10%, still more preferably at least 20%, and most preferably at least 30% or more of the administered particles preferably undergo apical to basolateral transcytosis from the pulmonary lumen.

In certain embodiments, the tumor in a subject is a primary tumor, such as that of the lung; however, the tumor in a subject may be a secondary tumor, such as a pulmonary metastasis from a primary tumor that is not of the lung. In various embodiments, the primary tumor is selected from the group consisting of a sarcoma, an adenocarcinoma, a choriocarcinoma, and a melanoma. In other embodiments, the tumor is a colon adenocarcinoma, a breast adenocarcinoma, an Ewing's sarcoma, or an osteosarcoma. For example, the primary tumor may be a renal cell carcinoma and the secondary tumor a tumor of the lung. In various embodiments, the clinical presentation of the pulmonary metastasis is a solitary metastasis, a cannonball, a lymphangitis carcinoimatosa, or a pleural effusion. A “primary” tumor is the original tumor in a subject. A “secondary” tumor is a cancer that has metastasized from the organ in which it first appeared to another organ.

Cell proliferation disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, adenocarcenomas, and leukemias.

Cancers of any organ can be treated, such as cancers of the colon, pancreas, breast, prostate, bone, liver, kidney, lung, testes, skin, pancreas, stomach, colorectal cancer, renal cell carcinoma, hepatocellular carcinoma, melanoma, etc.

Examples of breast cancer include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma. Examples of brain cancers include, but are not limited to, brain stem and hypophtalmic glioma, cerebellar and cerebral astrocytoma, medulloblastoma, ependymoma, as well as neuroectodermal and pineal tumor. Tumors of the male reproductive organs include, but are not limited to, prostate and testicular cancer. Tumors of the female reproductive organs include, but are not limited to, endometrial, cervical, ovarian, vaginal, and vulvar cancer, as well as sarcoma of the uterus. Tumors of the digestive tract include, but are not limited to, anal, colon, colorectal, esophageal, gallbladder, gastric, pancreatic, rectal, small-intestine, and salivary gland cancers. Tumors of the urinary tract include, but are not limited to, bladder, penile, kidney, renal pelvis, ureter, and urethral cancers. Eye cancers include, but are not limited to, intraocular melanoma and retinoblastoma. Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to, squamous cell carcinoma, Kaposi's sarcoma, malignant melanoma, Merkel cell skin cancer, and non-melanoma skin cancer. Head-and-neck cancers include, but are not limited to, laryngeal, hypopharyngeal, nasopharyngeal, and/or oropharyngeal cancers, and lip and oral cavity cancer. Lymphomas include, but are not limited to, AIDS-related lymphoma, non-Hodgkin's lymphoma, cutaneous T-cell lymphoma, Hodgkin's disease, and lymphoma of the central nervous system. Sarcomas include, but are not limited to, sarcoma of the soft tissue, osteosarcoma, malignant fibrous histiocytoma, lymphosarcoma, and rhabdomyosarcoma. Leukemias include, but are not limited to, acute myeloid leukemia, acute lymphoblastic leukemia, chronic lymphocytic leukemia, chronic myelogenous leukemia, and hairy cell leukemia. In addition to reducing the proliferation of tumor cells and inducing apoptosis, the particles of the invention can also cause tumor regression, e.g., a decrease in the size of a tumor, or in the extent of cancer in the body.

In addition to chemotherapeutic agents, the methods and compositions of the subject invention can incorporate treatments and agents utilizing, for example, angiogenesis inhibitors (Thalidomide, Bevacizumab), Bcl-2 antisense oligonucleotides (G3139), a PSA based vaccine, a PDGF receptor inhibitor (Gleevec), microtubule stabilizers (Epothilones), and a pro-apoptotic agent (Perifosine). Thus, the particles of the invention can be administered to a subject in combination (simultaneously or consecutively) with other agents useful for treating cell proliferation disorders (including polynucleotides encoding such agents) or other disorders. Likewise, the pharmaceutical compositions of the subject invention can include such agents (including polynucleotides encoding such agents).

The term “polynucleotide”, as used herein, refers to a polymeric form of nucleotides of any length, either ribonucleotides or deoxyribonucleotides. This term refers only to the primary structure of the molecule. Thus, the term includes double-stranded and single-stranded DNA, as well as double-stranded and single-stranded RNA. Thus, the term includes DNA, RNA, or DNA-DNA, DNA-RNA, or RNA-RNA hybrids, or protein nucleic acids (PNAs) formed by conjugating bases to an amino acid backbone. It also includes modifications, such as by methylation and/or by capping, and unmodified forms of the polynucleotide. The nucleotides may be synthetic, or naturally derived, and may contain genes, portions of genes, or other useful polynucleotides. In one embodiment, the polynucleotide comprises DNA containing all or part of the coding sequence for a polypeptide, or a complementary sequence thereof, such as interferon and/or IFN-inducible molecule. An encoded polypeptide may be intracellular, i.e., retained in the cytoplasm, nucleus, or in an organelle, or may be secreted by the cell. For secretion, the natural signal sequence present in a polypeptide may be retained. When the polypeptide or peptide is a fragment of a protein, a signal sequence may be provided so that, upon secretion and processing at the processing site, the desired protein will have the natural sequence. Specific examples of coding sequences of interest for use in accordance with the present invention include the polypeptide-coding sequences disclosed herein. The polynucleotides may also contain, optionally, one or more expressible marker genes for expression as an indication of successful transfection and expression of the nucleic acid sequences contained therein.

According to the present invention, an isolated nucleic acid molecule or nucleic acid sequence is a nucleic acid molecule or sequence that has been removed from its natural milieu. As such, “isolated” does not necessarily reflect the extent to which the nucleic acid molecule has been purified.

The terms “polypeptide” and “protein” are used interchangeably herein and indicate a molecular chain of amino acids of any length linked through peptide bonds. Thus, peptides, oligopeptides, and proteins are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

The particles of the present invention are useful as vectors for the delivery of polynucleotides encoding interferon (such as IFN-gamma) and/or an interferon-inducible molecule (such as 2-5 AS or IRF-1) to hosts in vitro or in vivo. The term “vector” is used to refer to any molecule (e.g., nucleic acid or plasmid) usable to transfer a polynucleotide, such as coding sequence information (e.g., nucleic acid sequence encoding a protein or other polypeptide), to a host cell. A vector typically includes a replicon in which another polynucleotide segment is attached, such as to bring about the replication and/or expression of the attached segment. The term includes expression vectors, cloning vectors, and the like. Thus, the term includes gene expression vectors capable of delivery/transfer of exogenous nucleic acid sequences into a host cell. The term “expression vector” refers to a vector that is suitable for use in a host cell (e.g., a subject's cell, tissue culture cell, cells of a cell line, etc.) and contains nucleic acid sequences which direct and/or control the expression of exogenous nucleic acid sequences. Expression includes, but is not limited to, processes such as transcription, translation, and RNA splicing, if introns are present. Nucleic acid sequences can be modified according to methods known in the art to provide optimal codon usage for expression in a particular expression system. The vector of the present invention may include elements to control targeting, expression and transcription of the nucleic acid sequence in a cell selective manner as is known in the art. The vector can include a control sequence, such as a promoter for controlling transcription of the exogenous material and can be either a constitutive or inducible promoter to allow selective transcription. The expression vector can also include a selection gene.

Each particle of the invention comprises a polynucleotide that is a coding sequence for an interferon, IFN-inducible molecule, or both. A “coding sequence” is a polynucleotide sequence that is transcribed into mRNA and/or translated into a polypeptide. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences. Variants or analogs may be prepared by the deletion of a portion of the coding sequence, by insertion of a sequence, and/or by substitution of one or more nucleotides within the sequence. For example, the particles of the present invention may be used to deliver coding sequences for interferon gamma, or variants or analogs thereof. Techniques for modifying nucleotide sequences, such as site-directed mutagenesis, are well known to those skilled in the art (See, e.g., Sambrook et al., Molecular Cloning: A Laboratory Manual, Second Edition, 1989; DNA Cloning, Vols. I and II, D. N. Glover ed., 1985). Optionally, the polynucleotides used in the particles of the present invention, and composition and methods of the invention that utilize such particles, can include non-coding sequences.

The term “operably-linked” is used herein to refer to an arrangement of flanking control sequences wherein the flanking sequences so described are configured or assembled so as to perform their usual function. Thus, a flanking control sequence operably-linked to a coding sequence may be capable of effecting the replication, transcription and/or translation of the coding sequence under conditions compatible with the control sequences. For example, a coding sequence is operably-linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence, and the promoter sequence can still be considered “operably-linked” to the coding sequence. Each nucleotide sequence coding for a polypeptide will typically have its own operably-linked promoter sequence. The promoter can be a constitutive promoter, or an inducible promoter to allow selective transcription. Optionally, the promoter can be a cell-specific or tissue-specific promoter. Promoters can be chosen based on the cell-type or tissue-type that is targeted for delivery or treatment, for example.

Suitable promoters include any that are known in the art or yet to be identified that will cause expression of interferon-encoding nucleic acid sequences or IFN-inducible molecule-encoding nucleic acid sequences in mammalian cells. Suitable promoters and other regulatory sequences can be selected as is desirable for a particular application. The promoters can be inducible, tissue-specific, or event-specific, as necessary. For example, the cytomegalovirus (CMV) promoter (Boshart et al., Cell, 1985, 41:521-530) and SV40 promoter (Subramani et al., Mol. Cell. Biol., 1981, 1:854-864) have been found to be suitable, but others can be used as well. Optionally, the polynucleotide used in the particles of the subject invention includes a sequence encoding a signal peptide upstream of the interferon-encoding and/or IFN-inducible molecule-encoding sequence, thereby permitting secretion of the interferon and/or IFN-inducible molecule from a host cell. Also, various promoters may be used to limit the expression of the polypeptide in specific cells or tissues, such as lung cells.

A tissue-specific and/or event-specific promoter or transcription element that responds to the target microenviroment and physiology can also be utilized for increased transgene expression at the desired site. There has been an immense amount of research activity directed at strategies for enhancing the transcriptional activity of weak tissue-specific promoters or otherwise increasing transgene expression with vectors. It is possible for such strategies to provide enhancement of gene expression equal to one or two orders of magnitude, for example (see Nettelbeck et al., Gene Ther., 1998, 5(12):1656-1664 and Qin et al., Hum. Gene Ther., 1997, 8(17):2019-2019). Examples of cardiac-specific promoters are the ventricular form of MLC-2v promoter (see, Zhu et al., Mol. Cell Biol., 1993, 13:4432-4444, Navankasattusas et al., Mol. Cell Biol., 1992, 12:1469-1479, 1992) and myosin light chain-2 promoter (Franz et al., Circ. Res., 1993, 73:629-638). The E-cadherin promoter directs expression specific to epithelial cells (Behrens et al., PNAS, 1991, 88:11495-11499), while the estrogen receptor (ER) 3 gene promoter directs expression specifically to the breast epithelium (Hopp et al., J. Mammary Gland Biol. Neoplasia, 1998, 3:73-83). The human C-reactive protein (CRP) gene promoter (Ruther et al., Oncogene 8:87-93, 1993) is a liver-specific promoter. An example of a muscle-specific gene promoter is human enolase (ENO3) (Peshavaria et al., Biochem. J., 1993, 292(Pt 3):701-704). A number of brain-specific promoters are available such as the thy-1 antigen and gamma-enolase promoters (Vibert et al., Eur. J. Biochem. 181:33-39, 1989). The prostate-specific antigen promoter provides prostate tissue specificity (Pang et al., Gene Ther., 1995, 6(11):1417-1426; Lee et al., Anticancer Res., 1996, 16(4A):1805-1811). The surfactant protein B promoter provides lung specificity (Strayer et al., Am. J. Respir. Cell Mol. Biol., 1998, 18(1):1-11). Any of the aforementioned promoters may be selected for targeted or regulated expression of the interferon-encoding and/or IFN-inducible protein-encoding polynucleotide.

The particles of the present invention can be targeted through various means. As indicated above, tissue-specific promoters or event-specific promoters may be utilized with polynucleotides encoding interferon and/or IFN-inducible molecules to further optimize and localize expression at target sites, such as within diseased tissues (e.g., cancer cells or tissues containing cancer cells). Robson et al. review various methodologies and vectors available for delivering and expressing a polynucleotide in vivo for the purpose of treating cancer (Robson, T. Hirst, D. G., J. Biomed. and Biotechnol., 2003, 2003(2):110-137, which is hereby incorporated by reference herein in its entirety). Among the various targeting techniques available, transcriptional targeting using tissue-specific and event-specific transcriptional control elements is discussed. For example, Table 1 at page 112 of the Robson et al. publication lists several tissue-specific promoters useful in cancer therapy. Tables 2-4 of the Robson et al. publication list tumor-specific promoters, tumor environment-specific promoters, and exogenously controlled inducible promoters, many of which were available at the time the patent application was filed. The successful delivery and expression of the p53 tumor suppressor gene in vivo has been documented (Horowitz, J. Curr. Opin. Mol. Ther., 1999, 1(4):500-509; Von Gruenigen, V. E. et al. Int. J. Gynecol. Cancer, 1999, 9(5):365-372; Fujiwara, T. et al., Mol. Urol., 2000, 4(2):51-54, respectively).

Many techniques for delivery of drugs and proteins are available in the art to reduce the effects of enzymatic degradation, to facilitate cell uptake, and to reduce any potential toxicity to normal (undiseased) cells, etc. Such methods and reagents can be utilized for administration of particles of the invention and their polynucleotide cargo to cells in vitro or in vivo. For example, peptides known as “cell penetrating peptides” (CPP) or “protein transduction domains” (PTD) have an ability to cross the cell membrane and enter the cell. PTDs can be linked to a cargo moiety such as a drug, peptide, or full-length protein, and can transport the moiety across the cell membrane. One well characterized PTD is the human immunodeficient virus (HIV)-1 Tat peptide (see, for example, Frankel et al., U.S. Pat. Nos. 5,804,604; 5,747,641; 6,674,980; 5,670,617; and 5,652,122; Fawell, S. et al., Proc. Natl. Acad. Sci. U.S.A., 1994, 91:664-668). Peptides such as the homeodomain of Drosophila antennapedia (ANTp) and arginine-rich peptides display similar properties (Derossi, D. et al., J. Biol. Chem., 1994, 269:10444-10450; Derossi, D. et al., Trends Cell Biol., 1998, 8:84-87; Rojas, M. et al., Nat. Biotechnol., 1998, 16:370-375; Futaki, S. et al., J. Biol. Chem., 2001, 276:5836-5840). VP22, a tegument protein from Herpes simplex virus type 1 (HSV-1), also has the ability to transport proteins across a cell membrane (Elliot et al., Cell, 1997, 88:223-233; Schwarze S. R. et al., Trends Pharmacol. Sci., 2000, 21:45-48). A common feature of these carriers is that they are highly basic and hydrophilic (Schwarze S. R. et al., Trends Cell Biol., 2000, 10:290-295). Coupling of these carriers to marker proteins such as beta-galactosidase has been shown to confer efficient internalization of the marker protein into cells. More recently, chimeric, in-frame fusion proteins containing these carriers have been used to deliver proteins to a wide spectrum of cell types both in vitro and in vivo. For example, VP22-p53 chimeric protein retained its ability to spread between cells and its pro-apoptotic activity, and had a widespread cytotoxic effect in p53 negative human osteosarcoma cells in vitro (Phelan, A. et al., Nature Biotechnol., 1998, 16:440-443). Intraperitoneal injection of the beta-galactosidase protein fused to the HIV-1 Tat peptide resulted in delivery of the biologically active fusion protein to all tissues in mice, including the brain (Schwarze S. R. et al., Science, 1999, 285:1569-1572).

Liposomes of various compositions can also be used for site-specific delivery of proteins and drugs (Witschi, C. et al., Pharm. Res., 1999, 16:382-390; Yeh, M. K. et al., Pharm. Res., 1996, 1693-1698). The interaction between the liposomes and their cargo usually relies on hydrophobic interactions or charge attractions, particularly in the case of cationic lipid delivery systems (Zelphati, O. et al., J. Biol. Chem., 2001, 276:35103-35110). Tat peptide-bearing liposomes have also been constructed and used to deliver cargo directly into the cytoplasm, bypassing the endocytotic pathway (Torchilin V. P. et al., Biochim. Biophys. Acta-Biomembranes, 2001, 1511:397-411; Torchilin V. P. et al., Proc. Natl. Acad. Sci. USA, 2001, 98:8786-8791). When encapsulated in sugar-grafted liposomes, pentamidine isethionate and a derivative have been found to be more potent in comparison to normal liposome-encapsulated drug or to the free drug (Banerjee, G. et al., J. Antimicrob. Chemother., 1996, 38(1):145-150). A thermo-sensitive liposomal taxol formulation (heat-mediated targeted drug delivery) has been administered in vivo to tumor-bearing mice in combination with local hyperthermia, and a significant reduction in tumor volume and an increase in survival time was observed compared to the equivalent dose of free taxol with or without hyperthermia (Sharma, D. et al., Melanoma Res., 1998, 8(3):240-244). Topical application of liposome preparations for delivery of insulin, IFN-alpha, IFN-gamma, and prostaglandin E1 have met with some success (Cevc G. et al., Biochim. Biophys, Acta, 1998, 1368:201-215; Foldvari M. et al., J. Liposome Res., 1997, 7:115-126; Short S. M. et al., Pharm. Res., 1996, 13:1020-1027; Foldvari M. et al., Urology, 1998, 52(5):838-843; U.S. Pat. No. 5,853,755).

Antibodies represent another targeting device that may make particle uptake tissue-specific or cell-specific (Mastrobattista, E. et al., Biochim. Biophys. Acta, 1999, 1419(2):353-363; Mastrobattista, E. et al., Adv. Drug Deliv. Rev., 1999, 40(1-2):103-127). The liposome approach offers several advantages, including the ability to slowly release encapsulated drugs and proteins, the capability of evading the immune system and proteolytic enzymes, and the ability to target tumors and cause preferentially accumulation in tumor tissues and their metastases by extravasation through their leaky neovasculature. Other carriers have also been used to deliver anti-cancer drugs to neoplastic cells, such as polyvinylpyrrolidone nanoparticles and maleylated bovine serum albumin (Sharma, D. et al., Oncol. Res., 1996, 8(7-8):281-286; Mukhopadhyay, A. et al., FEBS Lett., 1995, 376(1-2):95-98). Thus, using targeting and encapsulation technologies, which are very versatile and amenable to rational design and modification, delivery of particles of the invention to desired cells can be further facilitated.

As indicated above, the particles of the present invention can include a lipid component, such as a liposome. According to the present invention, a liposome comprises a lipid composition that is capable of fusing with the plasma membrane of a cell, thereby allowing the liposome to deliver a nucleic acid molecule and/or a protein composition into a cell. Some preferred liposomes include those liposomes commonly used in gene delivery methods known to those of skill in the art. Some preferred liposome delivery vehicles comprise multilamellar vesicle (MLV) lipids and extruded lipids, although the invention is not limited to such liposomes. Methods for preparation of MLVs are well known in the art. “Extruded lipids” are also contemplated. Extruded lipids are lipids that are prepared similarly to MLV lipids, but which are subsequently extruded through filters of decreasing size, as described in Templeton et al., Nature Biotech., 1997, 15:647-652, which is incorporated herein by reference in its entirety. Small unilamellar vesicle (SUV) lipids can also be used for preparing particles of the present invention. Other preferred liposome delivery vehicles comprise liposomes having a polycationic lipid composition (i.e., cationic liposomes). For example, cationic liposome compositions include, but are not limited to, any cationic liposome complexed with cholesterol, and without limitation, include DOTMA and cholesterol, DOTAP and cholesterol, DOTIM and cholesterol, and DDAB and cholesterol. Liposomes utilized in the present invention can be any size, including from about 10 to 1000 nanometers (nm), or any size in between.

A liposome delivery vehicle can be modified to target a particular site in a mammal, thereby targeting and making use of an interferon-encoding and/or IFN-inducible molecule-encoding nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. In some embodiments, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., Biochemistry, 1986, 25: 5500-6; Ho et al., J Biol Chem, 1987a, 262: 13979-84; Ho et al., J Biol Chem, 1987b, 262: 13973-8; and U.S. Pat. No. 4,957,735 to Huang et al., each of which is incorporated herein by reference in its entirety). In one embodiment, if avoidance of the efficient uptake of injected liposomes by reticuloendothelial system cells due to opsonization of liposomes by plasma proteins or other factors is desired, hydrophilic lipids, such as gangliosides (Allen et al., FEBS Lett, 1987, 223: 42-6) or polyethylene glycol (PEG)-derived lipids (Klibanov et al., FEBS Lett, 1990, 268: 235-7), can be incorporated into the bilayer of a conventional liposome to form the so-called sterically-stabilized or “stealth” liposomes (Woodle et al., Biochim Biophys Acta, 1992, 1113: 171-99). Variations of such liposomes are described, for example, in U.S. Pat. No. 5,705,187 to Unger et al., U.S. Pat. No. 5,820,873 to Choi et al., U.S. Pat. No. 5,817,856 to Tirosh et al.; U.S. Pat. No. 5,686,101 to Tagawa et al.; U.S. Pat. No. 5,043,164 to Huang et al., and U.S. Pat. No. 5,013,556 to Woodle et al., all of which are incorporated herein by reference in their entireties).

The size of the particle provides another means for targeting the particles of the invention to cells or tissues. For example, relatively small particles of the invention can be made to efficiently target ischemic tissue and tumor tissue, as described in U.S. Pat. No. 5,527,538, and U.S. Pat. Nos. 5,019,369, 5,435,989 and 5,441,745, the contents of which are hereby incorporated by reference in their entirety.

The particles of the invention can be targeted according to the mode of administration. For example, lung or other respiratory epithelial tissue can be targeted by intranasal administration, cervical cells can be targeted by intravaginal administration, and prostate tumors can be targeted by intrarectal administration. Skin cancer can be targeted by topical administration. Depending on location, tumors can be targeted locally, such as by injection, into the tumor mass.

Particles of the invention can be targeted by incorporating a ligand such as an antibody, a receptor, or other compound known to target particles such as liposomes or other vesicles to various sites. The ligands can be attached to cationic lipids used to form the particles of the present invention, or to a neutral lipid such as cholesterol used to stabilize the particle. Ligands that are specific for one or more specific cellular receptor sites are attached to a particle to form a delivery vehicle that can be targeted with a high degree of specificity to a target cell population of interest.

Suitable ligands for use in the present invention include, but are not limited to, sugars, proteins such as antibodies, hormones, lectins, major histocompatibility complex (MHC), and oligonucleotides that bind to or interact with a specific site. An important criteria for selecting an appropriate ligand is that the ligand is specific and is suitably bound to the surface of the particles in a manner which preserves the specificity. For example, the ligand can be covalently linked to the lipids used to prepare the particles. Alternatively, the ligand can be covalently bound to cholesterol or another neutral lipid, where the ligand-modified cholesterol is used to stabilize the lipid monolayer or bilayer.

The terms “transfection” and “transformation” are used interchangeably herein to refer to the insertion of an exogenous polynucleotide into a host, irrespective of the method used for the insertion, the molecular form of the polynucleotide that is inserted, or the nature of the host (e.g., prokaryotic or eukaryotic). The insertion of a polynucleotide per se and the insertion of a plasmid or vector comprising the exogenous polynucleotide are included. The exogenous polynucleotide may be directly transcribed and translated by the host or host cell, maintained as a nonintegrated vector, for example, a plasmid, or alternatively, may be stably integrated into the host genome. The terms “administration” and “treatment” are used herein interchangeably to refer to transfection of hosts in vitro or in vivo, using particles of the present invention.

The term “wild-type” (WT), as used herein, refers to the typical, most common or conventional form as it occurs in nature.

Thus, the present invention includes methods of gene therapy whereby polynucleotides encoding the desired gene product (an interferon, such as interferon-gamma, an IFN-inducible molecule, or both) are delivered to a subject, and the polynucleotide is expressed in vivo. The term “gene therapy”, as used herein, includes the transfer of genetic material (e.g., polynucleotides) of interest into a host to treat or prevent a genetic or acquired disease or condition phenotype, or to otherwise express the genetic material such that the encoded product is produced within the host. The genetic material of interest can encode a product (e.g., a protein, polypeptide, peptide, or functional RNA) whose production in vivo is desired. For example, in addition to interferon and/or an IFN-inducible molecule, the genetic material can encode a hormone, receptor, enzyme, polypeptide or peptide, of therapeutic and/or diagnostic value. For a review see, in general, the text “Gene Therapy” (Advances in Pharmacology 40, Academic Press, 1997). The genetic material may encode a product normally found within the species of the intended host, or within a different species. For example, if the polynucleotide encodes interferon-gamma, the cytokine may be human interferon-gamma, or that of another mammal, for example, regardless of the intended host. Preferably, the polynucleotide encodes a product that is normally found in the species of the intended host. Alternatively, the genetic material may encode a novel product.

Two basic approaches to gene therapy have evolved: (1) ex vivo and (2) in vivo gene therapy. The methods of the subject invention encompass either or both. In ex vivo gene therapy, host cells are removed from a patient and, while being cultured, are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient.

In in vivo gene therapy, target host cells are not removed from the subject, rather the gene to be transferred is introduced into the cells of the recipient organism in situ, that is within the recipient. Alternatively, if the host gene is defective, the gene is repaired in situ.

The particle of the present invention is capable of delivery/transfer of heterologous nucleic acid sequences into a prokaryotic or eukaryotic host cell in vitro or in vivo. The particle may include elements to control targeting, expression and transcription of the nucleic acid sequence in a cell selective manner as is known in the art. It should be noted that often the 5′UTR and/or 3′UTR of the gene may be replaced by the 5′UTR and/or 3′UTR of other expression vehicles.

Optionally, the particles of the invention may have biologically active agents other than polynucleotides as a component of the complex (either instead of, or in addition to, polynucleotides). Such biologically active agents include, but are not limited to, substances such as proteins, polypeptides, antibodies, antibody fragments, lipids, carbohydrates, and chemical compounds such as pharmaceuticals. The substances can be therapeutic agents, diagnostic materials, and/or research reagents.

The present invention includes pharmaceutical compositions comprising an effective amount of particles of the invention and a pharmaceutically acceptable carrier. The pharmaceutical compositions of the subject invention can be formulated according to known methods for preparing pharmaceutically useful compositions. As used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils.

The pharmaceutically acceptable carrier can be one adapted for a particular route of administration. For example, if the particles of the present invention are intended to be administered to the respiratory epithelium, a carrier appropriate for oral or intranasal administration can be used.

Formulations containing carriers are described in a number of sources which are well known and readily available to those skilled in the art. For example, Remington's Pharmaceutical Sciences (Martin E. W., 1995, Easton Pa., Mack Publishing Company, 19^{th} ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

As used herein, the terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, liver cancer, e.g., hepatic carcinoma, bladder cancer, colorectal cancer, endometrial carcinoma, kidney cancer, and thyroid cancer.

Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); peritoneal cancer; retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas.

As used herein, the term “tumor” refers to all neoplastic cell growth and proliferation, whether malignant or benign, and all pre-cancerous and cancerous cells and tissues. For example, a particular cancer may be characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site.

As used herein, the term “metastasis” refers to the process by which cancer cells are spread to distant parts of the body, such as from one organ and/or tissue to another not directly connected with it. The term is also used herein to refer to a tumor that develops through the metastatic process. Thus, as used herein, the term “metastasis” refers to neoplastic cell growth (e.g., tumor cell growth) in an unregulated fashion and spread to distal tissues and organs of the body. As used herein, the phrase “inhibiting metastasis” refers to the particles slowing and/or preventing metastasis or the spread of neoplastic cells to a site remote from the primary growth area.

The term “anti-cancer activity”, in reference to the particles of the invention, is intended to mean an activity which is able to substantially inhibit, slow, interfere, suppress, prevent, delay and/or arrest a cancer and/or a metastasis thereof (such as initiation, growth, spread, and/or progression thereof of such cancer and/or metastasis).

As used herein, the term “growth inhibitory amount” refers to an amount which inhibits growth of a target cell, such as a tumor cell, either in vitro or in vivo, irrespective of the mechanism by which cell growth is inhibited. In a preferred embodiment, the growth inhibitory amount inhibits growth of the target cell in cell culture by greater than about 20%, preferably greater than about 50%, most preferably greater than about 75% (e.g., from about 75% to about 100%).

The therapeutic methods of the invention can be advantageously combined with at least one additional therapeutic technique, including but not limited to chemotherapy, radiation therapy, surgery (e.g., surgical excision of cancerous or pre-cancerous cells), or any other therapy known to those of skill in the art of the treatment and management of cancer, such as administration of an anti-cancer agent.

As used herein, the term “anti-cancer agent” refers to a substance or treatment that inhibits the function of cancer cells, inhibits their formation, and/or causes their destruction in vitro or in vivo. Examples include, but are not limited to, cytotoxic agents (e.g., 5-fluorouracil, TAXOL) and anti-signaling agents (e.g., the PI3K inhibitor LY).

As used herein, the term “cytotoxic agent” refers to a substance that inhibits or prevents the function of cells and/or causes destruction of cells in vitro and/or in vivo. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, toxins such as small molecule toxins or enzymatically active toxins of bacterial, fungal, plant or animal origin, and antibodies, including fragments and/or variants thereof.

As used herein, the term “chemotherapeutic agent” is a chemical compound useful in the treatment of cancer, such as, for example, taxanes, e.g., paclitaxel (TAXOL, BRISTOL-MYERS SQUIBB Oncology, Princeton, N.J.) and doxetaxel (TAXOTERE, Rhone-Poulenc Rorer, Antony, France), chlorambucil, vincristine, vinblastine, anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston), and anti-androgens such as flutamide, nilutamide, bicalutamide, leuprolide, and goserelin, etc.

As used herein, the term “anti-signaling agent” refers to agents that interfere with cancer cell malignancy by inhibiting specific aberrant signal transduction circuits in the cell in vitro and/or in vivo. The PI3K inhibitor LY is an example of an anti-signalling agent.

The terms “comprising”, “consisting of” and “consisting essentially of” are defined according to their standard meaning. The terms may be substituted for one another throughout the instant application in order to attach the specific meaning associated with each term.

As used in this specification and the appended claims, the singular forms “a”, “an”, and “the” include plural reference unless the context clearly dictates otherwise. Thus, for example, a reference to “a particle” includes more than one such particle, a reference to “a polynucleotide” includes more than one such polynucleotide, a reference to “a polypeptide” includes more than one such polypeptide, a reference to “a host cell” includes more than one such host cell, a reference to an interferon or IFN-inducible molecule includes more than one such interferon or IFN-inducible molecule, and so forth.

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al. (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659; and 5,272,057; and incorporated herein by reference. Polymerase chain reaction (PCR) was carried out generally as in PCR Protocols: A Guide To Methods And Applications, Academic Press, San Diego, Calif. (1990). In situ (In-cell) PCR in combination with Flow Cytometry can be used for detection of cells containing specific DNA and mRNA sequences (Testoni et al., Blood, 1996, 87:3822).

It should be understood that the examples and embodiments described herein are for illustrative purposes only and that various modifications or changes in light thereof will be suggested to persons skilled in the art and are to be included within the spirit and purview of this application.

### EXAMPLE 1

### pIFN-γ Induces Apoptosis of HEp-2 Carcinoma Cells

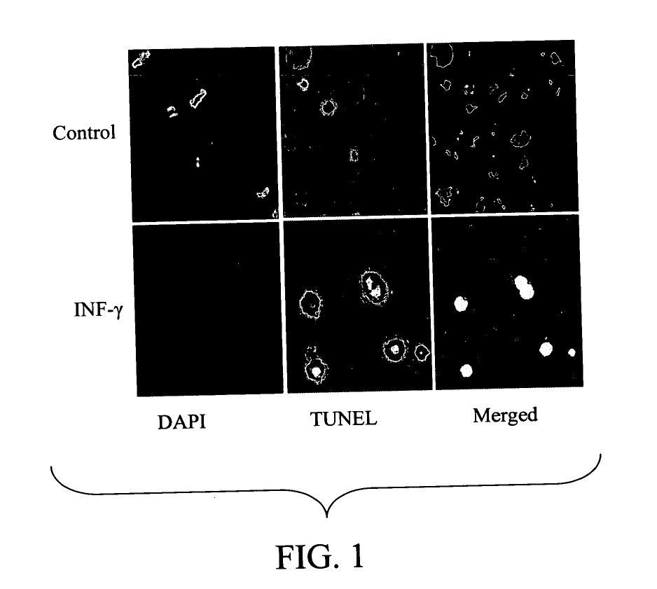

To determine the effect of overexpression of pIFN-γ on proliferation of A549 lung epithelial cells, cells were transfected with either pIFN-γ or empty vector, pVAX (control). Cell cycle analysis was performed using propidium iodide (PI) staining and flow cytometry 48 hours after transfection. No significant difference was observed between control and pIFN-γ-transfected cells in S1, Go-G1 and G2-M stages of the cell cycle (data not shown). However, an analysis of apoptosis using fluorescence microscopy cells transfected with pIFN-γ exhibited significantly higher apoptosis compared to cells transfected with either the control plasmid or a plasmid encoding pVAX (shown in FIG. 1).

Cells were seeded into 4-well slide dishes at 104 cells per well and allowed to grow to 75% confluency. Cells were treated for 20 hours with 1000 U/ml IFN-γ. After 24 hours, cells were fixed with 4% paraformaldehyde in PBS for 25 minutes at 4° C. and then permeabilized. Apoptotic cells were identified using a fluorescein-based, terminal nucleotidyl end-labeling kit (PROMEGA TUNEL Apoptosis Assay) that adds fluorescein conjugated dUTP to the 3′-hydroxyl ends of DNA fragments arising from apoptosis. After the reaction, the cells were rinsed in 2× saline citrate buffer and the nuclei were stained with DAPI. Stained cells were examined by immuno-fluorescence microscopy to determine the extent of apoptosis.

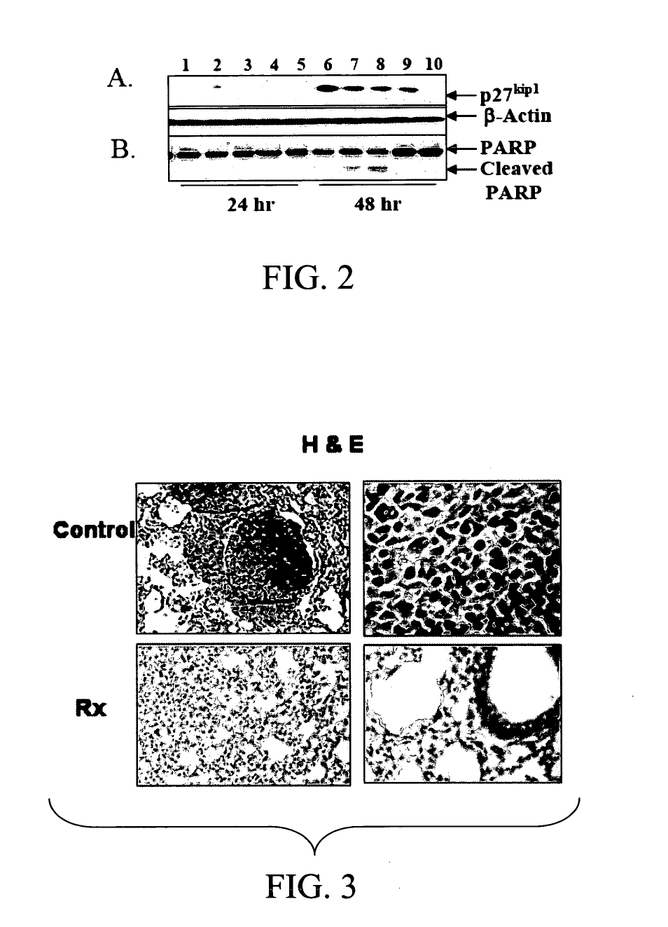

FIG. 2 demonstrates the detection of p27kip expression and PARP cleavage in IFN-gamma treated HEp-2 cells. Total cell extracts of HEp-2 cells (1×10⁶) treated as above were prepared after 24 and 48 hours of treatment and proteins were subjected to SDS-PAGE and western blotting was done with a monoclonal antibody to p27 kip1 (A) or an antibody to PARP (B). The lanes are as follows: 1) Untreated cells, 2) IFN-gamma 100 u/ml, 3) IFN-gamma 1000 u/ml, 4) IFN-beta 100 u/ml, 5) IFN-beta 1000 u/ml, 6) untreated cells, 7) IFN-gamma 100 u/ml, 8) IFN-gamma 1000 u/ml, 9) IFN-beta 100 u/ml, and 10) IFN-beta 1000 u/ml.

The apoptosis was confirmed by analysis of PARP cleavage in these cells 48 hours after transfection, which was significantly more prominent in pIFN-γ transfected cells (FIG. 2). Thus, pIFN-γ induces apoptosis of lung adenocarcinoma cells. Together, these studies indicate that pIFN-γ is an inducer of apoptosis in A549 lung adenocarcinoma cells.

### EXAMPLE 2

### Microarray Analysis of Chitosan—pIFNgamma Treated Lungs

Using MU11KsubA and B chips (Affymetrix), which contain probes interrogating about 11,000 murine genes and ESTs (Unigene, Build-4), as well as EST clusters from TIGR (1.0 Beta), we have identified a total of 126 differentially expressed genes whose expression level is altered in the CIN treated mouse lung in the range of −10.6- to 152.4-fold. A noteworthy observation is the up-regulation of the expression of a number of IFN-inducible genes, immune response related genes, and genes involved in signal transduction, including STAT1 and STAT4.

TABLE 1
GENE EXPRESSION ANALYSIS IN
BALB/c LUNG BY MICROARRAY

| | Max. Fold | |
|---|---|---|
| Category of Genes | Change | Genes |
| IFN-regulated | 12 | IFN-induced 15 KDa protein, |
| | | IFN-activable protein 204, |
| | | Eukaryotic initiation factor |
| | | 5, Mx protein, MIG, IP-10, |
| | | Interferon regulatory factor |
| | | (mirf7), interferon-activatable |
| | | protein, IFN-induced protein |
| | | 6-16 precursor, IFN-induced |
| | | guanylate-binding protein, |
| | | HLA-associated protein i |
| | | (phapi) 2′-5′ oilgo A synthetase |
| Immune-related | 8.4 | T-cell specific protein, |
| | | RegIII gamma protein, MHC |
| | | class II, MIP, Down regu- |
| | | latory protein (rpt-1r) of |
| | | interlukin-2, T-cell receptor |
| | | alpha-chain precursor, Immune- |
| | | responsive gene 1 (Irg1), |
| | | High affinity IgG receptor, |
| | | MHC, class III antigene |
| | | factor B, MEL-14, Lymphotoxin- |
| | | beta, C-11, Rantes, MAMA |
| | | Serine protease, Proteasome |
| | | subunit (Imp7) |
| Signal transduction | 10.8* | PDGF, GTPase IGTP, Gluco- |
| | | corticoid-attenuated |
| | | response gene 16, Stat1, |
| | | purine nucleotide binding |
| | | protein, G-protein-like |
| | | LRG-47, ras-related protein |
| | | ora2, GTP binding protein |
| | | (IRG-47), Stat 4, cathespin |
| | | s precursor, Oct binding |
| | | factor 1 (OBF-1), FYN binding |
| | | protein, High mobility group 2 |

The RNA was isolated from BALB/c lungs following 5 days of CIN treatment.
The mouse chips A and B, a total of 11,000 genes, were scanned.
The asterisk indicates the fold increase was uncertain, as no expression was observed in control lungs.
The genes are listed in no particular order.

### EXAMPLE 3

### Chitosan-Conjugated pIFN-γ Plasmid Prevents Metastasis of Lung Tumors in Nude Mice

BALB/c nude mice were injected intravenously with 5×10⁶ A549 cells, then treated one day afterwards and at weekly intervals with pIFN-γ or control plasmid. After 4 weeks, mice were examined for lung histology. The control animals showed tumors, whereas no tumors were seen in the pIFN-γ-treated group (FIG. 3). These results indicate that pIFN-γ has the potential to decrease tumor metastasis.

The results indicated in FIG. 3 were obtained when BALB/c nude mice were injected with A549 cells (5×10⁶ cells/mouse) intravenously and one group treated with pIFN-gamma and another group with pVAX as control. The lungs of control mice showed numerous lung nodules in contrast to mice treated with pIFN-gamma, which showed very few tumors. The lungs were removed from mice treated with nanoparticles carrying empty plasmid pVAX (control) or with pIFN-gamma (Rx) and H & E stained. The lungs of control mice showed numerous lung nodules with typical tumor cell morphology in contrast to mice treated with pIFN-gamma, which showed very few tumors.

### EXAMPLE 4

### Development of Thermogel from Modified Chitosan that Provides Sustained Release

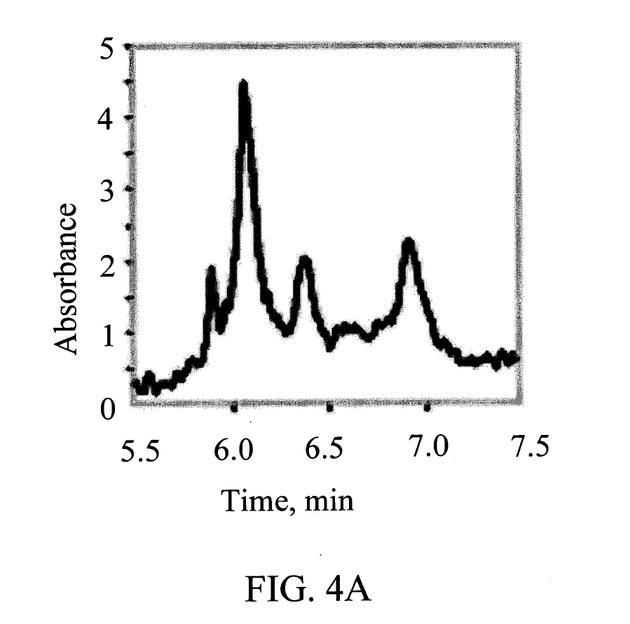

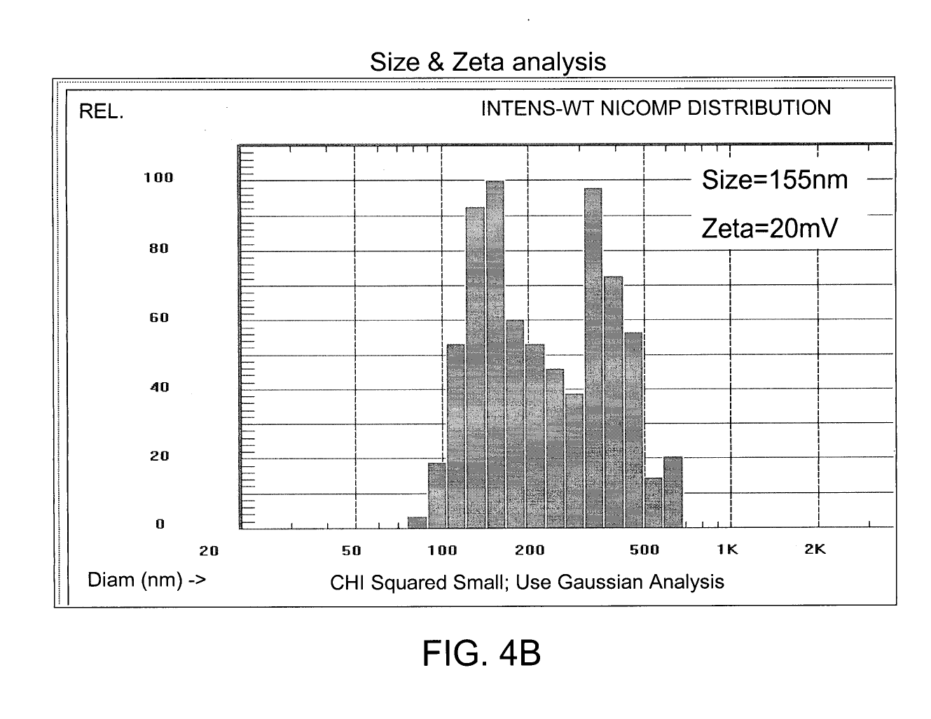

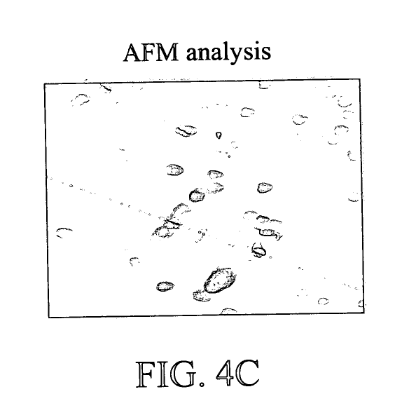

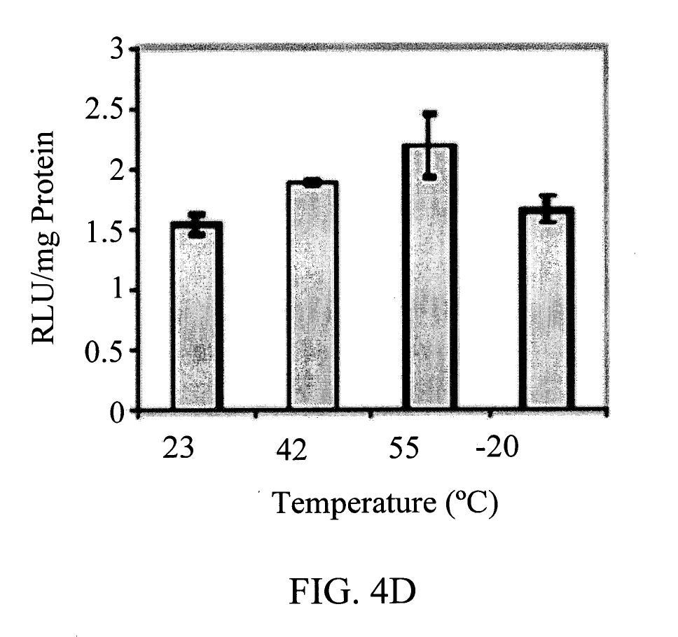

Using depolymerization methods, four novel chitosan derivative was synthesized. The products were separated by capillary gel electrophoresis. The plot shows the separation of 2 low molecular weight components (FIG. 4A). Nanogene-042(NG042) is a unique low molecular weight chitosan-based carrier, which has a particle size of 155 nm (major peak, with some aggregates at 335 nm), a zeta potential of about +20 mV with typical oligomeric structure, as identified by atomic force microscopy, and significant heat-stable properties for gene transfer, with both in vitro and in vivo expression (FIGS. 4B and 4C). Lyophilzed and resuspended NG042 particles retain functionality at ambient temperatures of 23° to 55° C. Nanogene complexes of pGL3 (firefly luciferase, Promega) was lyophilized, reconstituted with water and treated for 24 hours at RT (23° C.), 42° C., 55° C., and −20° C. A549 cells were plated and transfected with the above complexes. Uptake and expression of DNA was allowed to occur for 24 hours. Luciferase activity was determined by using Promega's Dual Assay kit. Readings were normalized to relative luminiscence units (RLU) per mg protein.

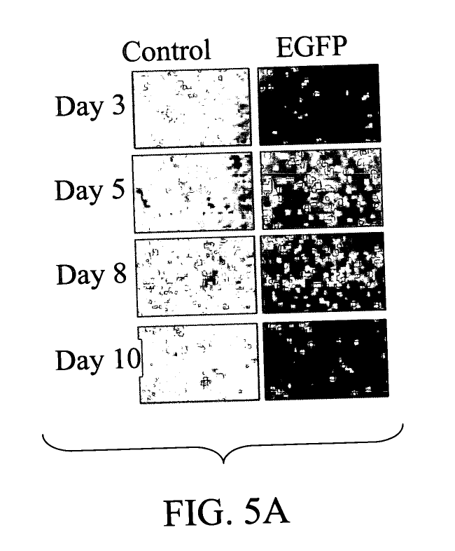

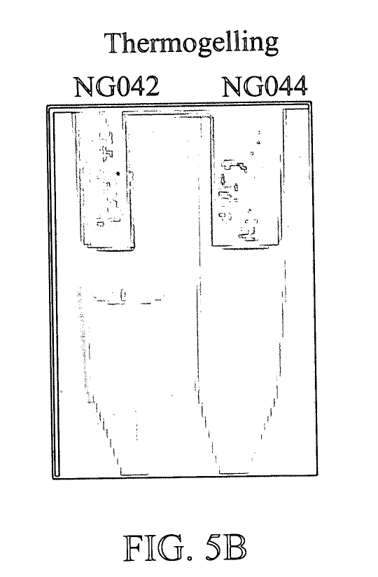

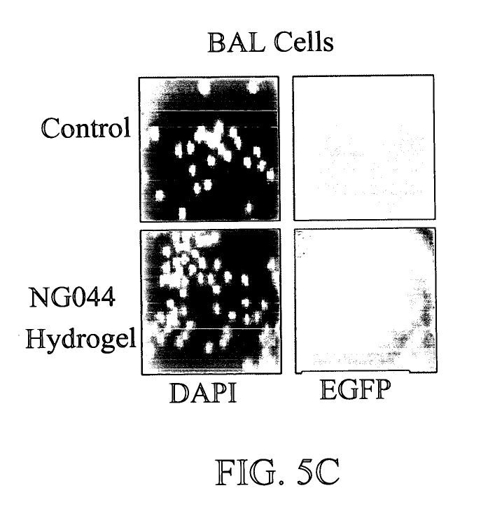

Another carrier, Nanogene-044 (NG044), is soluble in water and supports sustained gene expression in vivo (FIG. 5A). It also exhibits thermo-gelling properties, i.e., it is liquid at room temperature and forms a gel at temperatures above 37° C. (FIG. 5B). NG045 is a 1000-dalton oligomeric chitosan that is water soluble and shows sustained drug delivery following

NG044 was found to form a gel upon reacting with 2-glycerol phosphate, while NG042, another depolymerized chitosan does not.

To establish length of gene expression, Nanogene 044 (NG044) particles were complexed with DNA (5:1) encoding green fluorescent protein and a hydrogel was formed. The hydrogel was administered intranasally to groups of mice (n=4). Mice were sacrificed on the indicated days and broncho-alveolar lavage cells were examined by fluorescent microscopy. Another group received NG044 with pEGFP without gelling (Control). Gene expression in the mouse lung was measured by EGFP expression in BAL cells 10 and 20 days after administration. The results at day 10 were similar (not shown) for control and hydrogel, whereas after 20 days, mice given hydrogel continued EGFP show expression and no expression was detected in control mice.

Overall, the notion of intranasal chitosan nanoparticles carrying pIFN-γ for the treatment of cancer is based on the preliminary results that pIFN-γ may induce epithelial cell production of NO, which is known to possess anti-tumor effects, apoptosis of carcinoma cells, and abrogation of lung nodule formation in a murine model of lung metastasis. It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

All patents, patent applications, provisional applications, and publications (including information associated with sequence accession numbers) referred to or cited herein are incorporated by reference in their entirety, including all figures and tables, to the extent they are not inconsistent with the explicit teachings of this specification.

1. A method for treating a cell proliferation disorder in a subject, comprising administering a therapeutically effective amount of particles to the subject, wherein the particles comprise: a polynucleotide encoding an interferon, an interferon-inducible molecule, or both; and a chitin-containing component associated with the polynucleotide, wherein the polynucleotide is expressed in the subject and cell proliferation is reduced.

2. The method of claim 1, wherein the interferon is selected from the group consisting of alpha interferon, beta interferon, gamma interferon, omega interferon, and lambda interferon, or a biologically active fragment or derivative thereof.

3. The method of claim 1, wherein the interferon is gamma interferon.

4. The method of claim 1, wherein the interferon is a hybrid interferon.

5. The method of claim 1, wherein the interferon inducible molecule comprises interferon regulatory factor-1 (IRF-1).

6. The method of claim 1, wherein the interferon-inducible molecule comprises 2′-5′ oligoadenylate synthetase, interferon regulatory factor-1 (IRF-1), or both.

7. The method of claim 1, wherein the interferon-inducible molecule comprises a catalytically active subunit of 2′-5′ oligoadenylate synthetase selected from the group consisting of p40, p69, and p100 subunit.

8. The method of claim 1, wherein the 2′-5′ oligoadenylate synthetase comprises at least one splice variant selected from the group consisting of 40 kDa, 42 kDa, 46 kDa, 69 kDa, and 71 kDa.

9. The method of claim 1, wherein the chitin-containing component comprises chitosan or a chitosan derivative.

10. The method of claim 1, wherein the particles further comprise a lipid component associated with the chitin-containing component and the polynucleotide.

11. The method of claim 1, wherein the cell proliferation disorder is a cancer of the respiratory tract.

12. The method of claim 1, wherein the cell proliferation disorder is lung cancer.

13. The method of claim 1, wherein the particles are administered to the subject via a mucosal route.

14. The method of claim 1, wherein the particles are administered to the subject intranasally.

15. The method of claim 1, wherein the particles are administered to the subject as a spray, drops, powder, gel, or a combination of two or more of the foregoing.

16. The method of claim 1, wherein the subject is human.

17. The method of claim 1, wherein the subject is suffering from a cell proliferation disorder.

18. The method of claim 1, wherein the subject has been diagnosed with the cell proliferation disorder prior to said administering.

19. A method of inducing apoptosis in a cancer cell, comprising contacting a target cancer cell in vitro or in vivo with an effective amount of particles comprising: a polynucleotide encoding an interferon, an interferon-inducible molecule, or both; and a chitin-containing component associated with the polynucleotide, wherein the polynucleotide is expressed in the cancer cell and apoptosis is induced.

20. The method of claim 19, wherein the interferon is selected from the group consisting of alpha interferon, beta interferon, gamma interferon, omega interferon, and lambda interferon, or a biologically active fragment or derivative thereof.

21. The method of claim 19, wherein the interferon-inducible molecule comprises 2′-5′ oligoadenylate synthetase, interferon regulatory factor-1 (IRF-1), or both.

22. The method of claim 19, wherein the cancer cell is a respiratory epithelial cell.

23. A particle comprising a polynucleotide encoding an interferon, an interferon-inducible molecule, or both; and a chitin-containing component associated with the polynucleotide.

24. The particle of claim 23, wherein said chitin-containing component comprises chitosan or a chitosan derivative.

25. The particle of claim 23, wherein said particle further comprises a lipid component associated with the chitin-containing component and the polynucleotide.

26. The particle of claim 23, wherein said particle comprises a polynucleotide encoding said interferon and said interferon-inducible molecule.

27. The particle of claim 23, wherein said particle comprises a polynucleotide encoding said interferon and 2′-5′ oligoadenylate synthetase.

28. The particle of claim 23, wherein said particle comprises a polynucleotide encoding said interferon and interferon regulatory factor-1 (IRF-1).

29. The particle of claim 23, wherein said particle comprises a polynucleotide encoding 2′-5′ oligoadenylate synthetase and interferon regulatory factor-1 (IRF-1).

30. The particle of claim 23, wherein said particle comprises a polynucleotide encoding said interferon, 2′-5′ oligoadenylate synthetase, and interferon regulatory factor-1 (IRF-1).

31. The particle of claim 23, wherein said polynucleotide comprises one or more nucleotide sequences selected from the group consisting of SEQ ID NOs: 1, 3, 5, 7, 9, 11, 13, 14, 15, 16, 17, 19, 20, 21, 22, 23, 24, 25, 26, 27, 28, 29, 30, and 31.


SEQUENCE LISTING (31 sequences)

SEQ ID NO: 1 - DNA, 1203 bp, Homo sapiens
  CDS (1)..(1203)
  ref <301>: AUTHORS: Aissouni, Y., et al.
  ref <302>: TITLE: The cleavage/polyadenylation activity triggered by a U-rich motif sequence
  ref <303>: JOURNAL: J. Biol. Chem.
  ref <304>: VOLUME: 277
  ref <305>: ISSUE: 39
  ref <306>: PAGES: 35808-35814
  ref <307>: DATE: 2002
  ref <308>: DATABASE ACCESSION NUMBER: NCBI/NM_016816
  ref <309>: DATABASE ENTRY DATE: 2003-04-06
  ref <301>: AUTHORS: Behera, A.K., et al.
  ref <302>: TITLE: 2′-5′ Oligoadenylate synthesis plays a critical role in interferon-gamma inhibition
  ref <303>: JOURNAL: J. Biol. Chem.
  ref <304>: VOLUME: 277
  ref <305>: ISSUE: 28
  ref <306>: PAGES: 25601-25608
  ref <307>: DATE: 2002
  ref <308>: DATABASE ACCESSION NUMBER: NCBI/NM_016816
  ref <309>: DATABASE ENTRY DATE: 2003-04-06
  ref <301>: AUTHORS: Sarker, S.N., et al.
  ref <302>: TITLE: Identification of the substrate-binding sites of 2′-5′-oligoadenylate synthetase
  ref <303>: JOURNAL: J. Biol. Chem.
  ref <304>: VOLUME: 277
  ref <305>: ISSUE: 27
  ref <306>: PAGES: 24321-24330
  ref <307>: DATE: 2002
  ref <308>: DATABASE ACCESSION NUMBER: NCBI/NM_016816
  ref <309>: DATABASE ENTRY DATE: 2003-04-06
  ref <301>: AUTHORS: Hovnanian, A., et al.
  ref <302>: TITLE: The human 2′,5′-oligoadenylate synthetase locus is composed of three distinct genes
  ref <303>: JOURNAL: Genomics
  ref <304>: VOLUME: 52
  ref <305>: ISSUE: 3
  ref <306>: PAGES: 267-277
  ref <307>: DATE: 1998
  ref <308>: DATABASE ACCESSION NUMBER: NCBI/NM_016816
  ref <309>: DATABASE ENTRY DATE: 2003-04-06
  ref <301>: AUTHORS: Renault, B., et al.
  ref <302>: TITLE: A sequence-ready physical map of a region of 12q24.1
  ref <303>: JOURNAL: Genomics
  ref <304>: VOLUME: 45
  ref <305>: ISSUE: 2
  ref <306>: PAGES: 271-278
  ref <307>: DATE: 1997
  ref <308>: DATABASE ACCESSION NUMBER: NCBI/NM_016816
  ref <309>: DATABASE ENTRY DATE: 2003-04-06
  ref <301>: AUTHORS: Nechiporuk, T., et al.
  ref <302>: TITLE: A high-resolution PAC and BAC map of the SCA2 region
  ref <303>: JOURNAL: Genomics
  ref <304>: VOLUME: 44
  ref <305>: ISSUE: 3
  ref <306>: PAGES: 321-329
  ref <307>: DATE: 1997
  ref <308>: DATABASE ACCESSION NUMBER: NCBI/NM_016816
  ref <309>: DATABASE ENTRY DATE: 2003-04-06
  ref <301>: AUTHORS: Wathelet, M.G., et al.
  ref <302>: TITLE: Cloning and chromosomal location of human genes inducible by type I interferon
  ref <303>: JOURNAL: Somat. Cell Mol. Genet.
  ref <304>: VOLUME: 14
  ref <305>: ISSUE: 5
  ref <306>: PAGES: 415-426
  ref <307>: DATE: 1988
  ref <308>: DATABASE ACCESSION NUMBER: NCBI/NM_016816
  ref <309>: DATABASE ENTRY DATE: 2003-04-06
  ref <301>: AUTHORS: Rutherford, M.N., et al.
  ref <302>: TITLE: Interferon-induced binding of nuclear factors to promoter elements of the 2-5A synthetase gene
  ref <303>: JOURNAL: EMBO J.
  ref <304>: VOLUME: 7
  ref <305>: ISSUE: 3
  ref <306>: PAGES: 751-759
  ref <307>: DATE: 1988
  ref <308>: DATABASE ACCESSION NUMBER: NCBI/NM_016816
  ref <309>: DATABASE ENTRY DATE: 2003-04-06
  ref <301>: AUTHORS: Wathelet, M.G., et al.
  ref <302>: TITLE: New inducers revealed by the promoter sequence analysis of two interferon-activated human genes
  ref <303>: JOURNAL: Eur. J. Biochem.
  ref <304>: VOLUME: 169
  ref <305>: ISSUE: 2
  ref <306>: PAGES: 313-321
  ref <307>: DATE: 1987
  ref <308>: DATABASE ACCESSION NUMBER: NCBI/NM_016816
  ref <309>: DATABASE ENTRY DATE: 2003-04-06
  ref <301>: AUTHORS: Benech, P., et al.
  ref <302>: TITLE: Interferon-responsive regulatory elements in the promoter of the human 2′,5′-oligo(A) synthetase gene
  ref <303>: JOURNAL: Mol. Cell. Biol.
  ref <304>: VOLUME: 7
  ref <305>: ISSUE: 12
  ref <306>: PAGES: 4498-4504
  ref <307>: DATE: 1987
  ref <308>: DATABASE ACCESSION NUMBER: NCBI/NM_016816
  ref <309>: DATABASE ENTRY DATE: 2003-04-06
  ref <301>: AUTHORS: Hovanessian, A.G., et al.
  ref <302>: TITLE: Identification of 69-kd and 100-kd forms of 2-5A synthetase
  ref <303>: JOURNAL: EMBO J.
  ref <304>: VOLUME: 6
  ref <305>: ISSUE: 5
  ref <306>: PAGES: 1273-1280
  ref <307>: DATE: 1987
  ref <308>: DATABASE ACCESSION NUMBER: NCBI/NM_016816
  ref <309>: DATABASE ENTRY DATE: 2003-04-06
  ref <301>: AUTHORS: Williams, B.R., et al.
  ref <302>: TITLE: Interferon-regulated human 2-5A synthetase gene maps to chromosome
  ref <303>: JOURNAL: Somat. Cell Mol. Genet.
  ref <304>: VOLUME: 12
  ref <305>: ISSUE: 4
  ref <306>: PAGES: 403-408
  ref <307>: DATE: 1986
  ref <308>: DATABASE ACCESSION NUMBER: NCBI/NM_016816
  ref <309>: DATABASE ENTRY DATE: 2003-04-06
  ref <301>: AUTHORS: Shiojiri, S., et al.
  ref <302>: TITLE: Structure and expression of a cloned cDNA for human (2′-5′) oligoadenylate synthetase
  ref <303>: JOURNAL: J. Biochem.
  ref <304>: VOLUME: 99
  ref <305>: ISSUE: 5
  ref <306>: PAGES: 1455-1464
  ref <307>: DATE: 1986
  ref <308>: DATABASE ACCESSION NUMBER: NCBI/NM_016816
  ref <309>: DATABASE ENTRY DATE: 2003-04-06
  ref <301>: AUTHORS: Wathelet, M., et al.
  ref <302>: TITLE: Full-length sequence and expression of the 42 kDa 2-5A synthetase
  ref <303>: JOURNAL: FEBS Lett.
  ref <304>: VOLUME: 196
  ref <305>: ISSUE: 1
  ref <306>: PAGES: 113-120
  ref <307>: DATE: 1986
  ref <308>: DATABASE ACCESSION NUMBER: NCBI/NM_016816
  ref <309>: DATABASE ENTRY DATE: 2003-04-06
  ref <301>: AUTHORS: Benech, P., et al.
  ref <302>: TITLE: Structure of two forms of the interferon-induced (2′-5′) oligo A synthetase of human cells
  ref <303>: JOURNAL: EMBO J.
  ref <304>: VOLUME: 4
  ref <305>: ISSUE: 9
  ref <306>: PAGES: 2249-2256
  ref <307>: DATE: 1985
  ref <308>: DATABASE ACCESSION NUMBER: NCBI/NM_016816
  ref <309>: DATABASE ENTRY DATE: 2003-04-06
  ref <301>: AUTHORS: Saunders, M.E., et al.
  ref <302>: TITLE: Human 2-5A synthetase:  characterization of a novel cDNA and corresponding gene structure
  ref <303>: JOURNAL: EMBO J.
  ref <304>: VOLUME: 4
  ref <305>: ISSUE: 7
  ref <306>: PAGES: 1761-1768
  ref <307>: DATE: 1985
  ref <308>: DATABASE ACCESSION NUMBER: NCBI/NM_016816
  ref <309>: DATABASE ENTRY DATE: 2003-04-06
  ref <301>: AUTHORS: Merlin, G., et al.
  ref <302>: TITLE: Molecular cloning and sequence of partial cDNA for interferon-induced (2′-5′)oligo(A_ synthetase mRNA from human cells
  ref <303>: JOURNAL: Proc. Natl. Acad. Sci. U.S.A.
  ref <304>: VOLUME: 80
  ref <305>: ISSUE: 16
  ref <306>: PAGES: 4904-4908
  ref <307>: DATE: 1983
  ref <308>: DATABASE ACCESSION NUMBER: NCBI/NM_016816
  ref <309>: DATABASE ENTRY DATE: 2003-04-06
atgatggatctcagaaataccccagccaaatctctggacaagttcattgaagactatctcttgccagacacgtgtttccgcatgcaaatcgaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcactttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcagctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccaaggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctggctgaaagcaacagtacagacgatgagaccgacgatcccaggacgtatcagaaatatggttacattggaacacatgagtaccctcatttctctcatagacccagcacgctccaggcagcatccaccccacaggcagaagaggactggacctgcaccatcctctga

SEQ ID NO: 2 - protein, 400 aa, Homo sapiens
MMDLRNTPAKSLDKFIEDYLLPDTCFRMQIDHAIDIICGFLKERCFRGSSYPVCVSKVVKGGSSGKGTTLRGRSDADLVVFLSPLTTFQDQLNRRGEFIQEIRRQLEACQRERALSVKFEVQAPRWGNPRALSFVLSSLQLGEGVEFDVLPAFDALGQLTGSYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLAESNSTDDETDDPRTYQKYGYIGTHEYPHFSHRPSTLQAASTPQAEEDWTCTIL

SEQ ID NO: 3 - DNA, 1207 bp, Homo sapiens
  CDS (1)..(1206)
  ref <301>: AUTHORS: Benech et al.
  ref <302>: TITLE: Structure of two forms of the interferon-induced (2′-5′) oligo A synthetase human cells based on cDNAs and gene sequences
  ref <303>: JOURNAL: EMBO J.
  ref <304>: VOLUME: 4
  ref <305>: ISSUE: 9
  ref <306>: PAGES: 2249-2256
  ref <307>: DATE: 1985
  ref <308>: DATABASE ACCESSION NUMBER: NM_016816
  ref <309>: DATABASE ENTRY DATE: 2003-04-06
atgatggatctcagaaataccccagccaaatctctggacaagttcattgaagactatctcttgccagacacgtgtttccgcatgcaaatcgaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcactttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcagctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccaaggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctggctgaaagcaacagtacagacgatgagaccgacgatcccaggacgtatcagaaatatggttacattggaacacatgagtaccctcatttctctcatagacccagcacgctccaggcagcatccaccccacaggcagaagaggactggacctgcaccatcctctgaatgc

SEQ ID NO: 4 - protein, 400 aa, Homo sapiens; identical to SEQ ID NO: 2

SEQ ID NO: 5 - DNA, 2064 bp, Homo sapiens
  CDS (1)..(2064)
  ref <301>: AUTHORS: Hovnanian, A., et al.
  ref <302>: TITLE: The human 2′, 5′-oligoadenylate synthetase locus is comosed of three distinct genes
  ref <303>: JOURNAL: Genomics
  ref <304>: VOLUME: 52
  ref <305>: ISSUE: 3
  ref <306>: PAGES: 267-277
  ref <307>: DATE: 1998
  ref <308>: DATABASE ACCESSION NUMBER: NCBI/NM_002535
  ref <309>: DATABASE ENTRY DATE: 2003-04-03
  ref <301>: AUTHORS: Marie, I. and Hovanessian, A.G.
  ref <302>: TITLE: The 69-kDa 2-5A synthetase is composed of two homologous and adjacent functional domains
  ref <303>: JOURNAL: J. Biol. Chem.
  ref <304>: VOLUME: 267
  ref <305>: ISSUE: 14
  ref <306>: PAGES: 9933-9939
  ref <307>: DATE: 1992
  ref <308>: DATABASE ACCESSION NUMBER: NCBI/NM_002535
  ref <309>: DATABASE ENTRY DATE: 2003-04-03
  ref <301>: AUTHORS: Marie, I., et al.
  ref <302>: TITLE: Differential expression and distinct structure of 69- and 100-kDa forms of 2-5A synthetase
  ref <303>: JOURNAL: J. Biol. Chem.
  ref <304>: VOLUME: 265
  ref <305>: ISSUE: 30
  ref <306>: PAGES: 18601-18607
  ref <307>: DATE: 1990
  ref <308>: DATABASE ACCESSION NUMBER: NCBI/NM_002535
  ref <309>: DATABASE ENTRY DATE: 2003-04-03
  ref <301>: AUTHORS: Marie, I., et al.
  ref <302>: TITLE: Preparation and characterization of polyclonal antibodies specific for the 69 and 100 k-dalton forms of human 2-5A synthetase
  ref <303>: JOURNAL: Biochem. Biophys. Res. Commun.
  ref <304>: VOLUME: 160
  ref <305>: ISSUE: 2
  ref <306>: PAGES: 580-587
  ref <307>: DATE: 1989
  ref <308>: DATABASE ACCESSION NUMBER: NCBI/NM_002535
  ref <309>: DATABASE ENTRY DATE: 2003-04-03
  ref <301>: AUTHORS: Hovanessian, A.G., et al.
  ref <302>: TITLE: Characterization of 69- and 100-kDa forms of 2-5A-synthetase from interferon-treated human cells
  ref <303>: JOURNAL: J. Biol. Chem.
  ref <304>: VOLUME: 263
  ref <305>: ISSUE: 10
  ref <306>: PAGES: 4959
  ref <307>: DATE: 1988
  ref <308>: DATABASE ACCESSION NUMBER: NCBI/NM_002535
  ref <309>: DATABASE ENTRY DATE: 2003-04-03
  ref <301>: AUTHORS: Hovanessian, A.G., et al.
  ref <302>: TITLE: Identification of 69-kd and 100-kd forms of 2-5A synthesase
  ref <303>: JOURNAL: EMBO J.
  ref <304>: VOLUME: 6
  ref <305>: ISSUE: 5
  ref <306>: PAGES: 1273-1280
  ref <307>: DATE: 1987
  ref <308>: DATABASE ACCESSION NUMBER: NCBI/NM_002535
  ref <309>: DATABASE ENTRY DATE: 2003-04-03
atgggaaatggggagtcccagctgtcctcggtgcctgctcagaagctgggttggtttatccaggaatacctgaagccctacgaagaatgtcagacactgatcgacgagatggtgaacaccatctgtgacgtctgcaggaaccccgaacagttccccctggtgcagggagtggccataggtggctcctatggacggaaaacagtcttaagaggcaactccgatggtacccttgtccttttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggtccaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaacggttctggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgtcctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctcggcatcttctggaaggtcaattacaactttgaagatgagaccgtgaggaagtttctactgagccagttgcagaaaaccaggcctgtgatcttggacccaggcgaacccacaggtgacgtgggtggaggggaccgttggtgttggcatcttctggacaaagaagcaaaggttaggttatcctctccctgcttcaaggatgggactggaaacccaataccaccttggaaagtgccggtaaaagtcatctaa

SEQ ID NO: 6 - protein, 687 aa, Homo sapiens
MGNGESQLSSVPAQKLGWFIQEYLKPYEECQTLIDEMVNTICDVCRNPEQFPLVQGVAIGGSYGRKTVLRGNSDGTLVLFFSDLKQFQDQKRSQRDILDKTGDKLKFCLFTKWLKNNFEIQKSLDGSTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLGIFWKVNYNFEDETVRKFLLSQLQKTRPVILDPGEPTGDVGGGDRWCWHLLDKEAKVRLSSPCFKDGTGNPIPPWKVPVKVI

SEQ ID NO: 7 - DNA, 2186 bp, Homo sapiens
  ref <301>: AUTHORS: Marie, I. and Hovanessian, A.G.
  ref <302>: TITLE: The 69-kDa 2-5A synthetase is composed of two homologous and adjacent functional domains
  ref <303>: JOURNAL: J. Biol. Chem.
  ref <304>: VOLUME: 267
  ref <305>: ISSUE: 14
  ref <306>: PAGES: 9933-9939
  ref <307>: DATE: 1992
  ref <308>: DATABASE ACCESSION NUMBER: (unknown)
  ref <309>: DATABASE ENTRY DATE: 2003-04-03
atgggaaatggggagtcccagctgtcctcggtgcctgctcagaagctgggttggtttatccaggaatacctgaagccctacgaagaatgtcagacactgatcgacgagatggtgaacaccatctgtgacgtctgcaggaaccccgaacagttccccctggtgcagggagtggccataggtggctcctatggacggaaaacagtcttaagaggcaactccgatggtacccttgtccttttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggtccaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaacggttctggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgtcctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctcggcatcttctggaaggtcaattacaactttgaagatgagaccgtgaggaagtttctactgagccagttgcagaaaaccaggcctgtgatcttggacccaggcgaacccacaggtgacgtgggtggaggggaccgttggtgttggcatcttctggacaaagaagcaaaggttaggttatcctctccctgcttcaaggatgggactggaaacccaataccaccttggaaagtgccgacaatgcagacaccaggaagttgtggagctaggattccatcctattgtcaatgagatgttgtcatccagaagccatagaatcctgaataataattctaaaagaaacttctggagatcatctggcaatcgcttttaaa

SEQ ID NO: 8 - protein, 727 aa, Homo sapiens
MGNGESQLSSVPAQKLGWFIQEYLKPYEECQTLIDEMVNTICDVCRNPEQFPLVQGVAIGGSYGRKTVLRGNSDGTLVLFFSDLKQFQDQKRSQRDILDKTGDKLKFCLFTKWLKNNFEIQKSLDGSTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLGIFWKVNYNFEDETVRKFLLSQLQKTRPVILDPGEPTGDVGGGDRWCWHLLDKEAKVRLSSPCFKDGTGNPIPPWKVPTMQTPGSCGARIHPIVNEMFSSRSHRILNNNSKRNFTRSSGNRF

SEQ ID NO: 9 - DNA, 3264 bp, Homo sapiens
  CDS (1)..(3264)
  ref <301>: AUTHORS: Rebouillat, D., et al.
  ref <302>: TITLE: The 100-kDa 2′,5′-oligoadenylate synthetase catalyzing preferentially the synthesis of dimeric pppA2′p5′A molecules
  ref <303>: JOURNAL: J. Biol. Chem.
  ref <304>: VOLUME: 274
  ref <305>: ISSUE: 3
  ref <306>: PAGES: 1557-1565
  ref <307>: DATE: 1999
  ref <308>: DATABASE ACCESSION NUMBER: NCBI/AF_063613
  ref <309>: DATABASE ENTRY DATE: 1999-05-04
  ref <301>: AUTHORS: Rebouillat, D. and Hovanessian, A.G.
  ref <302>: TITLE: Direct Submission
  ref <303>: JOURNAL: Submitted (07-May-1998) Dept.of Aids and Retroviruses, Institut Pasteur
  ref <304>: VOLUME: 0
  ref <305>: ISSUE: 0
  ref <306>: PAGES: 0
  ref <307>: DATE: 1998
  ref <308>: DATABASE ACCESSION NUMBER: NCBI/AF_063613
  ref <309>: DATABASE ENTRY DATE: 1999-05-04
atggacttgtacagcaccccggccgctgcgctggacaggttcgtggccagaaggctgcagccgcggaaggagttcgtagagaaggcgcggcgcgctctgggcgccctggccgctgccctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccgcggtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctaggcgaaggcctccgaactgtcctgggcctgatccaacagcatcagcacctgtgtgttttctggactgtcaactatggcttcgaggaccctgcagttgggcagttcttgcagcggcacgttaagagacccaggcctgtgatcctggacccagctgaccccacatgggacctggggaatggggcagcctggcactgggatttgcatgcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactgcggagccagcatcgtacccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcacgtagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatgggagaatccccgcgtgctgagcttctcactgacatcccagacgatgctggaccagagtgtggactttgatgtgctgccagcctttgacgccctaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggcctatcatcctggatccggctgacccgacaggcaacctgggccacaatgcccgctgggacctgctggccaaggaagctgcagcctgcacatctgccctgtgctgcatgggacggaatggcatccccatccagccatggccagtgaaggctgctgtgtga

SEQ ID NO: 10 - protein, 1087 aa, Homo sapiens
MDLYSTPAAALDRFVARRLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSAVKPKPQVYSTLLNSGCQGGEHAACFTELRRNFVNIRPAKLKNLILLVKHWYHQVCLQGLWKETLPPVYALELLTIFAWEQGCKKDAFSLGEGLRTVLGLIQQHQHLCVFWTVNYGFEDPAVGQFLQRHVKRPRPVILDPADPTWDLGNGAAWHWDLHAQEAASCYDHPCFLRGMGDPVQSWKGPGLPRAGCSGLGHPIQLDPNQKTPENSKSLNAVYPRAGSKPPSCPAPGPTAEPASYPSVPGMALDLSQIPTKELDRFIQDHLKPSPQFQEQVKKAIDIILRCLHENCVHKASRVSKGGSFGRGTDLRDGCDVELIIFLNCFTDYKDQGPRRAEILDEMRAHVESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKGPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVKGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 11 - DNA, 1104 bp, Mus musculus
  CDS (1)..(1104)
  ref <301>: AUTHORS: Coccia, E.M., et al.
  ref <302>: TITLE: A full-length murine 2-5A synthetase cDNA transfected in NIH-3T3 cells
  ref <303>: JOURNAL: Virology
  ref <304>: VOLUME: 179
  ref <305>: ISSUE: 1
  ref <306>: PAGES: 228-233
  ref <307>: DATE: 1990
  ref <308>: DATABASE ACCESSION NUMBER: NCBI/M33863
  ref <309>: DATABASE ENTRY DATE: 1993-06-11
atggagcacggactcaggagcatcccagcctggacgctggacaagttcatagaggattacctccttcccgacaccacctttggtgctgatgtcaaatcagccgtcaatgtcgtgtgtgatttcctgaaggagagatgcttccaaggtgctgcccacccagtgagggtctccaaggtggtgaagggtggctcctcaggcaaaggcaccacactcaagggcaggtcagacgctgacctggtggtgttccttaacaatctcaccagctttgaggatcagttaaaccgacggggagagttcatcaaggaaattaagaaacagctgtacgaggttcagcatgagagacgttttagagtcaagtttgaggtccagagttcatggtggcccaacgcccggtctctgagcttcaagctgagcgccccccatctgcatcaggaggtggagtttgatgtgctgccagcctttgatgtcctgggtcatgttaatacttccagcaagcctgatcccagaatctatgccatcctcatcgaggaatgtacctccctggggaaggatggcgagttctctacctgcttcacggagctccagcggaacttcctgaagcagcgcccaaccaagctgaagagtctcatccgcctggtcaagcactggtaccaactgtgtaaggagaagctggggaagccattgcctccacagtacgccctagagttgctcactgtctttgcctgggaacaagggaatggatgttatgagttcaacacagcccagggcttccggaccgtcttggaactggtcatcaattatcagcatcttcgaatctactggacaaagtattatgactttcaacaccaggaggtctccaaatacctgcacagacagctcagaaaagccaggcctgtgatcctggacccagctgacccaacagggaatgtggccggtgggaacccagagggctggaggcggttggctgaagaggctgatgtgtggctatggtacccatgttttattaaaaaggatggttcccgagtgagctcctgggatgtgccgacggtggttcctgtaccttttgagcaggtagaagagaactggacatgtatcctgctgtga

SEQ ID NO: 12 - protein, 367 aa, Mus musculus
MEHGLRSIPAWTLDKFIEDYLLPDTTFGADVKSAVNVVCDFLKERCFQGAAHPVRVSKVVKGGSSGKGTTLKGRSDADLVVFLNNLTSFEDQLNRRGEFIKEIKKQLYEVQHERRFRVKFEVQSSWWPNARSLSFKLSAPHLHQEVEFDVLPAFDVLGHVNTSSKPDPRIYAILIEECTSLGKDGEFSTCFTELQRNFLKQRPTKLKSLIRLVKHWYQLCKEKLGKPLPPQYALELLTVFAWEQGNGCYEFNTAQGFRTVLELVINYQHLRIYWTKYYDFQHQEVSKYLHRQLRKARPVILDPADPTGNVAGGNPEGWRRLAEEADVWLWYPCFIKKDGSRVSSWDVPTVVPVPFEQVEENWTCILL

SEQ ID NO: 13 - DNA, 1590 bp, Homo sapiens
  ref <301>: AUTHORS: Aissouni, Y. et al.
  ref <302>: TITLE: The cleavage/polyadenylation activity triggered by a U-rich motif sequence
  ref <303>: JOURNAL: J. Biol. Chem.
  ref <304>: VOLUME: 277
  ref <305>: ISSUE: 39
  ref <306>: PAGES: 35808-35814
  ref <307>: DATE: 2002
  ref <308>: DATABASE ACCESSION NUMBER: NCBI/NM_016816
  ref <309>: DATABASE ENTRY DATE: 2003-04-06
  ref <301>: AUTHORS: Behera, A.K., et al.
  ref <302>: TITLE: 2′-5′ Oligoadenylate synthetase plays a critical role in interferon-gamma inhibition
  ref <303>: JOURNAL: J. Biol. Chem.
  ref <304>: VOLUME: 277
  ref <305>: ISSUE: 28
  ref <306>: PAGES: 25601-25608
  ref <307>: DATE: 2002
  ref <308>: DATABASE ACCESSION NUMBER: NCBI/NM_016816
  ref <309>: DATABASE ENTRY DATE: 2003-04-06
  ref <301>: AUTHORS: Sarkar, S.N., et al.
  ref <302>: TITLE: Identification of the substrate-binding sites of 2′-5′-oligoadenylate synthetase
  ref <303>: JOURNAL: J. Biol. Chem.
  ref <304>: VOLUME: 277
  ref <305>: ISSUE: 27
  ref <306>: PAGES: 24321-24330
  ref <307>: DATE: 2002
  ref <308>: DATABASE ACCESSION NUMBER: NCBI/NM_016816
  ref <309>: DATABASE ENTRY DATE: 2003-04-06
  ref <301>: AUTHORS: Hovnanian, A., et al.
  ref <302>: TITLE: The human 2′,5′-oligoadenylate synthetase locus is composed of three distinct genes
  ref <303>: JOURNAL: Genomics
  ref <304>: VOLUME: 52
  ref <305>: ISSUE: 3
  ref <306>: PAGES: 267-277
  ref <307>: DATE: 1998
  ref <308>: DATABASE ACCESSION NUMBER: NCBI/NM_016816
  ref <309>: DATABASE ENTRY DATE: 2003-04-06
  ref <301>: AUTHORS: Renault, B.
  ref <302>: TITLE: A sequence-ready physical map of a region of 12q24.1
  ref <303>: JOURNAL: Genomics
  ref <304>: VOLUME: 45
  ref <305>: ISSUE: 2
  ref <306>: PAGES: 271-278
  ref <307>: DATE: 1997
  ref <308>: DATABASE ACCESSION NUMBER: NCBI/NM_016816
  ref <309>: DATABASE ENTRY DATE: 2003-04-06
  ref <301>: AUTHORS: Nechiporuk, T., et al.
  ref <302>: TITLE: A high-resolution PAC and BAC map of the SCA2 region
  ref <303>: JOURNAL: Genomics
  ref <304>: VOLUME: 44
  ref <305>: ISSUE: 3
  ref <306>: PAGES: 321-329
  ref <307>: DATE: 1997
  ref <308>: DATABASE ACCESSION NUMBER: NCBI/NM_016816
  ref <309>: DATABASE ENTRY DATE: 2003-04-06
  ref <301>: AUTHORS: Wathelet, M.G., et al.
  ref <302>: TITLE: Cloning and chromosomal location of human genes inducible by type I interferon
  ref <303>: JOURNAL: Somat. Cell Mol. Genet.
  ref <304>: VOLUME: 14
  ref <305>: ISSUE: 5
  ref <306>: PAGES: 415-426
  ref <307>: DATE: 1988
  ref <308>: DATABASE ACCESSION NUMBER: NCBI/NM_016816
  ref <309>: DATABASE ENTRY DATE: 2003-04-06
  ref <301>: AUTHORS: Rutherford, M.N., et al.
  ref <302>: TITLE: Interferon-induced binding of nuclear factors to promoter elements of the 2-5A synthetase gene
  ref <303>: JOURNAL: EMBO J.
  ref <304>: VOLUME: 7
  ref <305>: ISSUE: 3
  ref <306>: PAGES: 751-759
  ref <307>: DATE: 1988
  ref <308>: DATABASE ACCESSION NUMBER: NCBI/NM_016816
  ref <309>: DATABASE ENTRY DATE: 2003-04-06
  ref <301>: AUTHORS: Wathelet, M.G., et al.
  ref <302>: TITLE: New inducers revealed by the promoter sequence analysis of two interferon-activated human genes
  ref <303>: JOURNAL: Eur. J. Biochem.
  ref <304>: VOLUME: 169
  ref <305>: ISSUE: 2
  ref <306>: PAGES: 313-321
  ref <307>: DATE: 1987
  ref <308>: DATABASE ACCESSION NUMBER: NCBI/NM_016816
  ref <309>: DATABASE ENTRY DATE: 2003-04-06
  ref <301>: AUTHORS: Benech, P., et al.
  ref <302>: TITLE: Interferon-responsive regulatory elements in the promoter of the human 2′,5′-oligo(A) synthetase gene
  ref <303>: JOURNAL: Mol. Cell. Biol.
  ref <304>: VOLUME: 7
  ref <305>: ISSUE: 12
  ref <306>: PAGES: 4498-4504
  ref <307>: DATE: 1987
  ref <308>: DATABASE ACCESSION NUMBER: NCBI/NM_016816
  ref <309>: DATABASE ENTRY DATE: 2003-04-06
  ref <301>: AUTHORS: Hovanessian, A.G., et al.
  ref <302>: TITLE: Identification of 69-kd and 100-kd forms of 2-5A sythetase
  ref <303>: JOURNAL: EMBO J.
  ref <304>: VOLUME: 6
  ref <305>: ISSUE: 5
  ref <306>: PAGES: 1273-1280
  ref <307>: DATE: 1987
  ref <308>: DATABASE ACCESSION NUMBER: NCBI/NM_016816
  ref <309>: DATABASE ENTRY DATE: 2003-04-06
  ref <301>: AUTHORS: Williams, B.R., et al.
  ref <302>: TITLE: Interferon-regulated human 2-5A synthetase gene maps to chromosome
  ref <303>: JOURNAL: Somat. Cell Mol. Genet.
  ref <304>: VOLUME: 12
  ref <305>: ISSUE: 4
  ref <306>: PAGES: 403-408
  ref <307>: DATE: 1986
  ref <308>: DATABASE ACCESSION NUMBER: NCBI/NM_016816
  ref <309>: DATABASE ENTRY DATE: 2003-04-06
  ref <301>: AUTHORS: Shiojiri, S., et al.
  ref <302>: TITLE: Structure and expression of a cloned cDNA
  ref <303>: JOURNAL: J. Biochem.
  ref <304>: VOLUME: 99
  ref <305>: ISSUE: 5
  ref <306>: PAGES: 1455-1464
  ref <307>: DATE: 1986
  ref <308>: DATABASE ACCESSION NUMBER: NCBI/NM_016816
  ref <309>: DATABASE ENTRY DATE: 2003-04-06
  ref <301>: AUTHORS: Wathelet, M., et al.
  ref <302>: TITLE: Full-length sequence and expression of the 42 kDa 2-5A synthetase
  ref <303>: JOURNAL: FEBS Lett.
  ref <304>: VOLUME: 196
  ref <305>: ISSUE: 1
  ref <306>: PAGES: 113-120
  ref <307>: DATE: 1986
  ref <308>: DATABASE ACCESSION NUMBER: NCBI/NM_016816
  ref <309>: DATABASE ENTRY DATE: 2003-04-06
  ref <301>: AUTHORS: Benech, P., et al.
  ref <302>: TITLE: Structure of two forms of the interferon-induced (2′-5′) oligo A synthetase
  ref <303>: JOURNAL: EMBO J.
  ref <304>: VOLUME: 4
  ref <305>: ISSUE: 9
  ref <306>: PAGES: 2249-2256
  ref <307>: DATE: 1985
  ref <308>: DATABASE ACCESSION NUMBER: NCBI/NM_016816
  ref <309>: DATABASE ENTRY DATE: 2003-04-06
  ref <301>: AUTHORS: Saunders, M.E., et al.
  ref <302>: TITLE: Human 2-5A synthetase:  characterization of a novel cDNA and corresponding gene structure
  ref <303>: JOURNAL: EMBO J.
  ref <304>: VOLUME: 4
  ref <305>: ISSUE: 7
  ref <306>: PAGES: 1761-1768
  ref <307>: DATE: 1985
  ref <308>: DATABASE ACCESSION NUMBER: NCBI/NM_016816
  ref <309>: DATABASE ENTRY DATE: 2003-04-06
  ref <301>: AUTHORS: Merlin, G., et al.
  ref <302>: TITLE: Molecular cloning and sequence of partial cDNA for interferon-induced (2′-5′) oligo(A) synthetase mRNA from human cells
  ref <303>: JOURNAL: Proc. Natl. Acad. Sci. U.S.A.
  ref <304>: VOLUME: 80
  ref <305>: ISSUE: 16
  ref <306>: PAGES: 4904-4908
  ref <307>: DATE: 1983
  ref <308>: DATABASE ACCESSION NUMBER: NCBI/NM_016816
  ref <309>: DATABASE ENTRY DATE: 2003-04-06
gaggcagttctgttgccactctctctcctgtcaatgatggatctcagaaataccccagccaaatctctggacaagttcattgaagactatctcttgccagacacgtgtttccgcatgcaaatcgaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcactttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcagctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccaaggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctggctgaaagcaacagtacagacgatgagaccgacgatcccaggacgtatcagaaatatggttacattggaacacatgagtaccctcatttctctcatagacccagcacgctccaggcagcatccaccccacaggcagaagaggactggacctgcaccatcctctgaatgccagtgcatcttgggggaaagggctccagtgttatctggaccagttccttcattttcaggtgggactcttgatccagagaagacaaagctcctcagtgagctggtgtataatccaagacagaacccaagtctcctgactcctggccttctatgccctctatcctatcatagataacattctccacagcctcacttcattccacctattctctgaaaatattccctgagagagaacagagagatttagataagagaatgaaattccagccttgactttcttctgtgcacctgatgggagggtaatgtctaatgtattatcaataacaataaaaataaagcaaataccaaaaa

SEQ ID NO: 14 - DNA, 3068 bp, Homo sapiens
  ref <301>: AUTHORS: Hovnanian, A., et al.
  ref <302>: TITLE: The human 2′,5′-oligoadenylate synthetase locus is composed of three distinct genes
  ref <303>: JOURNAL: Genomics
  ref <304>: VOLUME: 52
  ref <305>: ISSUE: 3
  ref <306>: PAGES: 267-277
  ref <307>: DATE: 1998
  ref <308>: DATABASE ACCESSION NUMBER: NCBI/NM_002535
  ref <309>: DATABASE ENTRY DATE: 2003-04-03
  ref <301>: AUTHORS: Marie, I. and Hovanessian, A.G.
  ref <302>: TITLE: The 69-kDa 2-5A synthetase is composed of two homologous and adjacent functional domains
  ref <303>: JOURNAL: J. Biol. Chem.
  ref <304>: VOLUME: 267
  ref <305>: ISSUE: 14
  ref <306>: PAGES: 9933-9939
  ref <307>: DATE: 1992
  ref <308>: DATABASE ACCESSION NUMBER: NCBI/NM_002535
  ref <309>: DATABASE ENTRY DATE: 2003-04-03
  ref <301>: AUTHORS: Marie, I., et al.
  ref <302>: TITLE: Differential expression and distinct structure of 69- and 100-kDa forms of 2-5A synthetase
  ref <303>: JOURNAL: J. Biol. Chem.
  ref <304>: VOLUME: 265
  ref <305>: ISSUE: 30
  ref <306>: PAGES: 18601-18607
  ref <307>: DATE: 1990
  ref <308>: DATABASE ACCESSION NUMBER: NCBI/NM_002535
  ref <309>: DATABASE ENTRY DATE: 2003-04-03
  ref <301>: AUTHORS: Marie, I., et al.
  ref <302>: TITLE: Preparation and characterization of polyclonal antibodies
  ref <303>: JOURNAL: Biochem. Biophys. Res. Commun.
  ref <304>: VOLUME: 160
  ref <305>: ISSUE: 2
  ref <306>: PAGES: 580-587
  ref <307>: DATE: 1989
  ref <308>: DATABASE ACCESSION NUMBER: NCBI/NM_002535
  ref <309>: DATABASE ENTRY DATE: 2003-04-03
  ref <301>: AUTHORS: Hovanessian, A.G., et al.
  ref <302>: TITLE: Characterization of 69- and 100-kDa forms of 2-5A-synthetase
  ref <303>: JOURNAL: J. Biol. Chem.
  ref <304>: VOLUME: 263
  ref <305>: ISSUE: 10
  ref <306>: PAGES: 4959
  ref <307>: DATE: 1988
  ref <308>: DATABASE ACCESSION NUMBER: NCBI/NM_002535
  ref <309>: DATABASE ENTRY DATE: 2003-04-03
  ref <301>: AUTHORS: Hovanessian, A.G., et al.
  ref <302>: TITLE: Identification of 69-kd and 100-kd forms of 2-5A synthetase
  ref <303>: JOURNAL: EMBO J.
  ref <304>: VOLUME: 6
  ref <305>: ISSUE: 5
  ref <306>: PAGES: 1273-1280
  ref <307>: DATE: 1987
  ref <308>: DATABASE ACCESSION NUMBER: NCBI/NM_002535
  ref <309>: DATABASE ENTRY DATE: 2003-04-03
cggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtctgcaggaaccccgaacagttccccctggtgcagggagtggccataggtggctcctatggacggaaaacagtcttaagaggcaactccgatggtacccttgtccttttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggtccaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaacggttctggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgtcctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctcggcatcttctggaaggtcaattacaactttgaagatgagaccgtgaggaagtttctactgagccagttgcagaaaaccaggcctgtgatcttggacccaggcgaacccacaggtgacgtgggtggaggggaccgttggtgttggcatcttctggacaaagaagcaaaggttaggttatcctctccctgcttcaaggatgggactggaaacccaataccaccttggaaagtgccggtaaaagtcatctaaaggaggcgttgtctggaaatagccctgtaacaggcttgaatcaaagaacttctcctactgtagcaacctgaaattaactcagacacaaataaaggaaacccagctcacaggagcttaaacagctggtcagcccccctaagcccccactacaagtgatcctcaggcaggtaaccccagattcatgcactgtagggctgggcgcagcatccctaggtctctacccagtagatgccactagccctcctctcccagtgacaaccaaaagtcttcacatgttcaaacgttcccctgggttcacagatctttctgcctttggcttttggctccaccctctttagctgttaatttgagtacttatggccctgaaagcggccacggtgcctccagatggcaggtttgcaatccaagcaggaagaaggaaaagatacccaaaggtcaagaacacagtgattttattagaagtttcatccgcaaattttcttccatttcattgctcagaatgtcatgtggttacctgtaacttgaaggtggctacaaagatgactgtggaggtggttgcacttgccacccaaggatgtctgccacacctctccaagccctcctacctaccaagatatacctgatatatccaccagatatctcctcagatatacttggttctctccaccaggttctttctttaaagcaggattctcaactttgatacttactcacattgggctagacagttctttgtttggaggctctcttgtgcatgtaggatgttgagcagcatgtgtggcctgtacccagtacatgccacccagttgtgacaattaaaagtgtcttgagactttatcatgtgtcttctgccctaggtgagaacccttgcactacaggaaccctacacccaacctggggggaatgtagggaagaggtgccaagccaaccgtggggttagctctaattattaagttatgcattataaataaataccaaaaaattg

SEQ ID NO: 15 - DNA, 6270 bp, Homo sapiens
  ref <301>: AUTHORS: Rebouillat, D., et al.
  ref <302>: TITLE: The 100-kDa 2′,5′-oligoadenylate synthetase catalyzing preferentially the synthesis of dimeric pppA2′p5′A molecules
  ref <303>: JOURNAL: J. Biol. Chem.
  ref <304>: VOLUME: 274
  ref <305>: ISSUE: 3
  ref <306>: PAGES: 1557-1565
  ref <307>: DATE: 1999
  ref <308>: DATABASE ACCESSION NUMBER: NCBI/AF063613
  ref <309>: DATABASE ENTRY DATE: 1999-05-04
  ref <301>: AUTHORS: Rebouillat, D., and Hovanessian, A.G.
  ref <302>: TITLE: Direct Submission
  ref <303>: JOURNAL: Submitted (07-May-1998) Dept. of AIDS and Retroviruses, Institut Pasteur
  ref <304>: VOLUME: 0
  ref <305>: ISSUE: 0
  ref <306>: PAGES: 0
  ref <307>: DATE: 1998
  ref <308>: DATABASE ACCESSION NUMBER: NCBI/AF063613
  ref <309>: DATABASE ENTRY DATE: 1999-05-04
gccctgcttccccttgcacctgcgccgggcggccatggacttgtacagcaccccggccgctgcgctggacaggttcgtggccagaaggctgcagccgcggaaggagttcgtagagaaggcgcggcgcgctctgggcgccctggccgctgccctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccgcggtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctaggcgaaggcctccgaactgtcctgggcctgatccaacagcatcagcacctgtgtgttttctggactgtcaactatggcttcgaggaccctgcagttgggcagttcttgcagcggcacgttaagagacccaggcctgtgatcctggacccagctgaccccacatgggacctggggaatggggcagcctggcactgggatttgcatgcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactgcggagccagcatcgtacccctctgtgccgggaatggccttggacctgtctcagatccccaccaaggagctggaccgcttcatccaggaccacctgaagccgagcccccagttccaggagcaggtgaaaaaggccatcgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcacgtagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatgggagaatccccgcgtgctgagcttctcactgacatcccagacgatgctggaccagagtgtggactttgatgtgctgccagcctttgacgccctaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggcctatcatcctggatccggctgacccgacaggcaacctgggccacaatgcccgctgggacctgctggccaaggaagctgcagcctgcacatctgccctgtgctgcatgggacggaatggcatccccatccagccatggccagtgaaggctgctgtgtgaagttgagaaaatcagcggtcctactggatgaagagaagatggacaccagccctcagcatgaggaaattcagggtcccctaccagatgagagagattgtgtacatgtgtgtgtgagcacatgtgtgcatgtgtgtgcacacgtgtgcatgtgtgtgttttagtgaatctgctctcccagctcacacactcccctgcctcccatggcttacacactaggatccagactccatggtttgacaccagcctgcgtttgcagcttctctgtcacttccatgactctatcctcataccaccactgctgcttcccacccagctgagaatgccccctcctccctgactcctctctgcccatgcaaattagctcacatctttcctcctgctgcaatccatcccttcctcccattggcctctccttgccaaatctaaatactttatatagggatggcagagagttcccatctcatctgtcagccacagtcatttggtactggctacctggagccttatcttctgaagggttttaaagaatggccaattagctgagaagaattatctaatcaattagtgatgtctgccatggatgcagtagaggaaagtggtggtacaagtgccatgattgattagcaatgtctgcactggatatggaaaaaagaaggtgcttgcaggtttacagtgtatatgtgggctattgaagagccctctgagctcggttgctagcaggagagcatgcccatattggcttactttgtctgccacagacacagacagagggagttgggacatgcatgctatggggaccctcttgttggacacctaattggatgcctcttcatgagaggcctccttttcttcaccttttatgctgcactcctcccctagtttacacatcttgatgctgtggctcagtttgccttcctgaatttttattgggtccctgttttctctcctaacatgctgagattctgcatccccacagcctaaactgagccagtggccaaacaaccgtgctcagcctgtttctctctgccctctagagcaaggcccaccaggtccatccaggaggctctcctgacctcaagtccaacaacagtgtccacactagtcaaggttcagcccagaaaacagaaagcactctaggaatcttaggcagaaagggattttatctaaatcactggaaaggctggaggagcagaaggcagaggccaccactggactattggtttcaatattagaccactgtagccgaatcagaggccagagagcagccactgctactgctaatgccaccactacccctgccatcactgccccacatggacaaaactggagtcgagacctaggttagattcctgcaaccacaaacatccatcagggatggccagctgccagagctgcgggaagacggatcccacctccctttcttagcagaatctaaattacagccagacctctggctgcagaggagtctgagacatgtatgattgaatgggtgccaagtgccagggggcggagtccccagcagatgcatcctggccatctgttgcgtggatgagggagtgggtctatctcagaggaaggaacaggaaacaaagaaaggaagccactgaacatcccttctctgctccacaggagtgtcttagacagcctgactctccacaaaccactgttaaaacttacctgctaggaatgctagattgaatgggatgggaagagccttccctcattattgtcattcttggagagaggtgagcaaccaagggaagctcctctgattcacctagaacctgttctctgccgtctttggctcagcctacagagactagagtaggtgaagggacagaggacagggcttctaatacctgtgccatattgacagcctccatccctgtcccccatcttggtgctgaaccaacgctaagggcaccttcttagactcacctcatcgatactgcctggtaatccaaagctagaactctcaggaccccaaactccacctcttggattggccctggctgctgccacacacatatccaagagctcagggccagttctggtgggcagcagagacctgctctgccaagttgtccagcagcagagtggccctggcctgggcatcacaagccagtgatgctcctgggaagaccaggtggcaggtcgcagttgggtaccttccattcccaccacacagactctgggcctccccgcaaaatggctccagaattagagtaattatgagatggtgggaaccagagcaactcaggtgcatgatacaaggagaggttgtcatctgggtagggcagagaggagggcttgctcatctgaacaggggtgtatttcattccaggccctcagtctttggcaatggccaccctggtgttggcatattggccccactgtaacttttgggggcttcccggtctagccacaccctcggatggaaagacttgactgcataaagatgtcagttctccctgagttgattgataggcttaatggtcaccctaaaaacacccacatatgcttttcgatggaaccagataagttgacgctaaagttcttatggaaaaatacacacgcaatagctaggaaaacacagggaaagaagagttctgagcagggcctagtcttagccaatattaaaacatactatgaagcctctgatacttaaacagcatggcgctggtacgtaaatagaccaatgcagttaggtggctctttccaagactctggggaaaaaagtagtaaaaagctaaatgcaatcaatcagcaattgaaagctaagtgagagagccagagggcctccttggtggtaaaagagggttgcatttcttgcagccagaaggcagagaaagtgaagaccaagtccagaactgaatcctaagaaatgcaggactgcaaagaaattggtgtgtgtgtgtgtgtgtgtgtgtgtgtgtgtttaatttttaaaaagtttttattcggaatccgcg

SEQ ID NO: 16 - DNA, 1412 bp, Mus musculus
  ref <301>: AUTHORS: Coccia, E.M., et al.
  ref <302>: TITLE: A full-length murine 2-5A synthetase cDNA transfected in NIH-3T3 cells
  ref <303>: JOURNAL: Virology
  ref <304>: VOLUME: 179
  ref <305>: ISSUE: 1
  ref <306>: PAGES: 228-233
  ref <307>: DATE: 1990
  ref <308>: DATABASE ACCESSION NUMBER: NCBI/M33863
  ref <309>: DATABASE ENTRY DATE: 1993-06-11
ccaggctgggagacccaggaagctccagacttagcatggagcacggactcaggagcatcccagcctggacgctggacaagttcatagaggattacctccttcccgacaccacctttggtgctgatgtcaaatcagccgtcaatgtcgtgtgtgatttcctgaaggagagatgcttccaaggtgctgcccacccagtgagggtctccaaggtggtgaagggtggctcctcaggcaaaggcaccacactcaagggcaggtcagacgctgacctggtggtgttccttaacaatctcaccagctttgaggatcagttaaaccgacggggagagttcatcaaggaaattaagaaacagctgtacgaggttcagcatgagagacgttttagagtcaagtttgaggtccagagttcatggtggcccaacgcccggtctctgagcttcaagctgagcgccccccatctgcatcaggaggtggagtttgatgtgctgccagcctttgatgtcctgggtcatgttaatacttccagcaagcctgatcccagaatctatgccatcctcatcgaggaatgtacctccctggggaaggatggcgagttctctacctgcttcacggagctccagcggaacttcctgaagcagcgcccaaccaagctgaagagtctcatccgcctggtcaagcactggtaccaactgtgtaaggagaagctggggaagccattgcctccacagtacgccctagagttgctcactgtctttgcctgggaacaagggaatggatgttatgagttcaacacagcccagggcttccggaccgtcttggaactggtcatcaattatcagcatcttcgaatctactggacaaagtattatgactttcaacaccaggaggtctccaaatacctgcacagacagctcagaaaagccaggcctgtgatcctggacccagctgacccaacagggaatgtggccggtgggaacccagagggctggaggcggttggctgaagaggctgatgtgtggctatggtacccatgttttattaaaaaggatggttcccgagtgagctcctgggatgtgccgacggtggttcctgtaccttttgagcaggtagaagagaactggacatgtatcctgctgtgagcacagcagcacctgcccaggagactgctggtcaggggcatttgctgctctgctgcaggcccatgacccagtgagggagggccccacctggcatcagactccgtgcttctgatgcctgccagccatgtttgactcctgtccaatcacagccagccttcctcaacagattcagaaggagaggaaagaacacacgcttggtgtccatctgtccacctgttggaaggttctgtctgacaaagtctgatcaacaataaaccacagcaggtgccgtca

SEQ ID NO: 17 - DNA, 1193 bp, Homo sapiens
tgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcaaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaact

SEQ ID NO: 18 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 19 - DNA, 838 bp, Homo sapiens
gagtctaactgcaacctttcgaagcctttgctctggcacaacaggtagtaggcgacactggtcgtgttgttgacatgaccaacaagtgtctcctccaaattgctctcctgttgtgcttctccacgacagctctttccatgagctacaacttgcttggattcctacaaagaagcagcaattgtcagtgtcagaagctcctgtggcaattgaatgggaggcttgaatactgcctcaaggacaggaggaactttgacatccctgaggagattaagcagctgcagcagttccagaaggaggacgccgcagtgaccatctatgagatgctccagaacatctttgctattttcagacaagattcatcgagcactggctggaatgagactattgttgagaacctcctggctaatgtctatcatcagagaaaccatctgaagacagtcctggaagaaaaactggagaaagaagatttcaccaggggaaaacgcatgagcagtctgcacctgaaaagatattatgggaggattctgcattacctgaaggccaaggaggacagtcactgtgcctggaccatagtcagagtggaaatcctaaggaacttttacgtcattaacagacttacaggttacctccgaaactgaagatctcctagcctgtgcctctgggacgggacaattgcttcaagcattcttcaaccagcagatgctgtttaagtgactgatggcgaatgtactgcatatgaaaggacactagaagattttgaaatttttattaaattatgaggtatttttatttatttaaattttattttggaaaataaattatttttggtgcaaaagtc

SEQ ID NO: 20 - DNA, 840 bp, Homo sapiens
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 21 - DNA, 588 bp, Homo sapiens
atggccttcgtgctctctctactcatggccctggtgctggtcagctacggcccaggaggatccctgggttgtgacctgtctcagaaccacgtgctggttggcaggaagaacctcaggctcctggacgaaatgaggagactctcccctcacttttgtctgcaggacagaaaagacttcgctttaccccaggaaatggtggagggcggccagctccaggaggcccaggccatctctgtgctccatgagatgctccagcagagcttcaacctcttccacacagagcactcctctgctgcctgggacaccaccctcctggagccatgccgcactggactccatcagcagctggacaacctggatgcctgcctggggcaggtgatgggagaggaagactctgccctgggaaggacgggccccaccctggctctgaagaggtacttccagggcatccatgtctacctgaaagagaagggatacagcgactgcgcctgggaaaccgtcagactggaaatcatgagatccttctcttcattaatcagcttgcaagaaaggttaagaatgatggatggagacctgagctcaccttga

SEQ ID NO: 22 - DNA, 961 bp, Homo sapiens
tgagcctaaaccttaggctcacccatttcaaccagtctagcagcatctgcaacatctacaatggccttgacctttgctttactggtggccctcctggtgctcagctgcaagtcaagctgctctgtgggctgtgatctgcctcaaacccacagcctgggtagcaggaggaccttgatgctcctggcacagatgaggaaaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgattttcattaattcgtatgccagctcacctttttatgatctgccatttcaaagactcatgtttctgctatgaccatgacacgatttaaatctttttcaaatgtttttaggagtattaatcaacattgtattcagctcttaaggcactagtcccttacagaggaccatgctgactgatccattatctatttaaatatttttaaaatattatttatttaactatttataaaacaacttatttttgttcatattacgtcatgtgcacctttgcacagtggttaatgtaataaaatatgttctttgtatttggtaa

SEQ ID NO: 23 - DNA, 737 bp, Homo sapiens
tacccacctcaggtagcctagtgatatttgcaaaatcccaatggcccggtccttttctttactgatggccgtgctggtactcagctacaaatccatctgctctctgggctgtgatctgcctcagacccacagcctgcgtaataggagggccttgatactcctggcacaaatgggaagaatctctcctttctcctgcttgaaggacagacatgaattcagattcccggaggaggagtttgatggccaccagttccagaagactcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggacttcatcctggctgtgaggaaatacttccaaagaatcactctttatctaacagagaagaaatacagcccttgtgcctgggaggttgtcagagcagaaatcatgagatccttctctttttcaacaaacttgaaaaaaggattaaggaggaaggattgaaaactggttcatcatggaaatgattctcattgactaatgcatcatctcacactttcatgagttcttccatttcaaagactcacttctataaccaccacaagttgaatcaaaatttccaaatgttttc

SEQ ID NO: 24 - DNA, 1933 bp, Homo sapiens
gatctggtaaacctgaagcaaatatagaaacctatagggcctgacttcctacataaagtaaggagggtaaaaatggaggctagaataagggttaaaattttgcttctagaacagagaaaatgatttttttcatatatatatgaatatatattatatatacacatatatacatatattcactatagtgtgtatacataaatatataatatatatattgttagtgtagtgtgtgtctgattatttacatgcatatagtatatacacttatgactttagtacccagacgtttttcatttgattaagcattcatttgtattgacacagctgaagtttactggagtttagctgaagtctaatgcaaaattaatagattgttgtcatcctcttaaggtcatagggagaacacacaaatgaaaacagtaaaagaaactgaaagtacagagaaatgttcagaaaatgaaaaccatgtgtttcctattaaaagccatgcatacaagcaatgtcttcagaaaacctagggtccaaggttaagccatatcccagctcagtaaagccaggagcatcctcatttcccaatggccctcctgttccctctactggcagccctagtgatgaccagctatagccctgttggatctctgggctgtgatctgcctcagaaccatggcctacttagcaggaacaccttggtgcttctgcaccaaatgaggagaatctcccctttcttgtgtctcaaggacagaagagacttcaggttcccccaggagatggtaaaagggagccagttgcagaaggcccatgtcatgtctgtcctccatgagatgctgcagcagatcttcagcctcttccacacagagcgctcctctgctgcctggaacatgaccctcctagaccaactccacactggacttcatcagcaactgcaacacctggagacctgcttgctgcaggtagtgggagaaggagaatctgctggggcaattagcagccctgcactgaccttgaggaggtacttccagggaatccgtgtctacctgaaagagaagaaatacagcgactgtgcctgggaagttgtcagaatggaaatcatgaaatccttgttcttatcaacaaacatgcaagaaagactgagaagtaaagatagagacctgggctcatcttgaaatgattctcattgattaatttgccatataacacttgcacatgtgactctggtcaattcaaaagactcttatttcggctttaatcacagaattgactgaattagttctgcaaatactttgtcggtatattaagccagtatatgttaaaaagacttaggttcaggggcatcagtccctaagatgttatttatttttactcatttatttattcttacattttatcatatttatactatttatattcttatataacaaatgtttgcctttacattgtattaagataacaaaacatgttcagctttccatttggttaaatattgtattttgttatttattaaattattttcaaacaaaacttcttgaagttatttattcgaaaaccaaaatccaaacactagttttctgaaccaaatcaaggaatggacggtaatatacacttacctattcattcattccatttacataatatgtataaagtgagtatcaaagtggcatattttggaattgatgtcaagcaatgcaggtgtactcattgcatgactgtatcaaaatatctcatgtaaccaataaatatatacacttactatgtatcccacaaaaattaaaaagttattttaaaaaagaaatacaggtgaataaacacagtttctttccgtgttgaagagctttcattcttacaggaaaagaaacagtaaagatgtaccaatttcgcttatatgaaacactacaaagataagtaaaagaaaatgatgttctcatactagaagctt

SEQ ID NO: 25 - DNA, 734 bp, Homo sapiens
tgggtgacagcctcagagtgtttcttctgctgacaaagaccagagatcaggaatgaaactagacatgactggggactgcacgccagtgctggtgctgatggccgcagtgctgaccgtgactggagcagttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctgaccctcccaccagtcatgcaacctgagattttatttataaattagccacttgtcttaatttattgccacccagtcgctat

SEQ ID NO: 26 - DNA, 594 bp, Homo sapiens
gacatgaccggggactgcatgccagtgctggtgctgatggccgcagtgctgaccgtgactggagcagttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 27 - DNA, 594 bp, Homo sapiens; identical to SEQ ID NO: 26

SEQ ID NO: 28 - DNA, 856 bp, Homo sapiens
aattaccttttcactttacacacatcatcttggattgcccattttgcgtggctaaaaagcagagccatgccgctggggaagcagttgcgatttagccatggctgcagcttggaccgtggtgctggtgactttggtgctaggcttggccgtggcaggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgtgtctgagaacgtcaacccaccctgagtccacctgacaccccacaccttatttatgcgctgagccctactccttccttaatttatttcctctcaccctttatttatgaagctgcagccctgactgagacatagggctgagtttattgttttacttttatacattatgcacaaataaacaacaaggaattgga

SEQ ID NO: 29 - DNA, 607 bp, Artificial Sequence
  Synthetic DNA encoding human interferon-like peptide
aattcaccgtgcgtgttgactattttacctctggcggtgataatgaaatcaattgtgagcggataacaattaaaaattaaggaggatcactatctgtgatttgccgcagactcatagcttcggcaaccgaacagcactcatcctcttggcccaattggcccaaatgggtcgcatttccctgttctcgtgccttaaagaccgccacgatttcgggtttccacaggaagagtttgacggcaatcagttccaaaaggctcaggcaatctcggttctgcatgagatgattcagcaaatcttcaacctgttctccactaaagactcttcggctgcttggaacgaatccttgcttgataaattctccactgaactctatcagcaactgaacgtacttgaagcttgcgttatccaggaggtaggcgttgaagagactccgcttatgaatgttgagtctatcctggctgtatctaagtattttcaccgaattaccctctacctcagtgagaagaaatattcaccgtgtgcgtgggaaattgtgagagccgaaatcatgcgttccctgactcttctgaccaacctccaggaacgcctgcgtaataaagactaatag

SEQ ID NO: 30 - DNA, 611 bp, Artificial Sequence
  Synthetic DNA encoding human interferon-like peptide
aattcaccgtgcgtgttgactattttacctctggcggtgataatgaaatcaattgtgagcggataacaattattacccaacttgaggaatttataatgtgtgatttaccacaaacccatagcttgggcaaccgaagagcactcatcctgttggcccaattggcccaaatgggtcgcatttccctgttctcgtgccttaaagaccgccacgatttcgggtttccacaggaagagtttgacggcaatcagttccaaaaggctcaggcaatctcggttctgcatgagatgattcagcaaatcttcaacctgttctccactaaagactgttcggctgcttggaacgaatccttgcttgataaattctccactgaactctatcagcaactgaacgtacttgaagcttgcgttatccaggaggtaggcgttgaagagactccgcttatgaatgttgagtctatcctggctgtatctaagtattttcaccgaattaccctctacctcagtgagaagaaatattcaccgtgtgcgtgggaaattgtgagagccgaaatctagcgttccctgactcttctgacgaacctccaggaacgcctgcgtaataaagactaatag

SEQ ID NO: 31 - DNA, 2035 bp, Homo sapiens
cgagccccgccgaaccgaggccacccggagccgtgcccagtccacgccggccgtgcccggcggccttaagaaccaggcaacctctgccttcttccctcttccactcggagtcgcgctccgcgcgccctcactgcagcccctgcgtcgccgggaccctcgcgcgcgaccagccgaatcgctcctgcagcagagccaacatgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtaaggaggagccagaaattgacagcccagggggggatattgggctgagtctacagcgtgtcttcacagatctgaagaacatggatgccacctggctggacagcctgctgaccccagtccggttgccctccatccaggccattccctgtgcaccgtagcagggcccctgggcccctcttattcctctaggcaagcaggacctggcatcatggtggatatggtgcagagaagctggacttctgtgggcccctcaacagccaagtgtgaccccactgccaagtggggatgggcctccctccttgggtcattgacctctcagggcctggcaggccagtgtctgggtttttcttgtggtgtaaagctggccctgcctcctgggaagatgaggttctgagaccagtgtatcaggtcagggacttggacaggagtcagtgtctggctttttcctctgagcccagctgcctggagagggtctcgctgtcactggctggctcctaggggaacagaccagtgaccccagaaaagcataacaccaatcccagggctggctctgcactaagcgaaaattgcactaaatgaatctcgttccaaagaactaccccttttcagctgagccctggggactgttccaaagccagtgaatgtgaaggaaactcccctccttcggggcaatgctccctcagcctcagaggagctctaccctgctccctgctttggctgaggggcttgggaaaaaaacttggcactttttcgtgtggatcttgccacatttctgatcagaggtgtacactaacatttcccccgagctcttggcctttgcatttatttatacagtgccttgctcggggcccaccaccccctcaagccccagcagccctcaacaggcccagggagggaagtgtgagcgccttggtatgacttaaaattggaaatgtcatctaaccattaagtcatgtgtgaacacataaggacgtgtgtaaatatgtacatttgtctttttataaaaagtaaaattgtt